(19) United States
(12) Patent Application Publication
(10) Pub. No.: US 2023/0028293 A1
(43) Pub. Date: Jan. 26, 2023

(54) MULTIPLE MYELOMA COMBINATION THERAPIES BASED ON PROTEIN TRANSLATION INHIBITORS AND IMMUNOMODULATORS

(71) Applicants: Daniel Sherbenou, Denver, CO (US); Zachary Walker, Aurora, CO (US); Beau Idler, Parker, CO (US); The Regents of the University of Colorado, a body corporate, Denver, CO (US)

(72) Inventors: Daniel Sherbenou, Denver, CO (US); Zachary Walker, Aurora, CO (US); Beau Idler, Parker, CO (US)

(21) Appl. No.: 17/778,347

(22) PCT Filed: Dec. 8, 2020
(86) PCT No.: PCT/US2020/063829

§ 371 Date: May 19, 2022

Related U.S. Application Data
(60) Provisional application No. 62/945,204, filed on Dec. 8, 2019.
(60) Provisional application No. 63/085,483, filed on Sep. 30, 2020.

(51) Int. Cl. A61K 31/566; A61K 31/573; A61K 31/69; A61K 31/55; A61K 31/454; A61P 35/00

(52) U.S. Cl. CPC A61K 31/566; A61K 31/573; A61K 31/69; A61K 31/55; A61K 31/454; A61P 35/00

(57) ABSTRACT

Multiple myeloma (MM) combination therapies based on protein translation inhibitors, immunomodulators, and bromodomain extra-terminal inhibitors. Methods are provided for treating multiple myeloma in a subject, including administering a therapeutically effective amount of at least one protein translation inhibitor and a therapeutically effective amount of at least one immunomodulatory drug (IMiD), administering a therapeutically effective amount of at least one protein translation inhibitor and a therapeutically effective amount of at least one BET inhibitor, and/or administering a therapeutically effective amount of at least one IMiD and a therapeutically effective amount of at least one BET inhibitor.

### STATEMENT REGARDING FEDERALLY SPONSORED RESEARCH

This invention was made with government support under Grant No. 1K08CA222704-01A1 awarded by the National Institutes of Health. The government has certain rights in the invention.

### FIELD

Embodiments disclosed herein provide methods for treating multiple myeloma using combination therapies based on protein translation inhibitors and immunomodulators.

### BACKGROUND

Multiple myeloma (MM) is an aggressive hematologic malignancy characterized by over-proliferation and tissue invasion by malignant plasma cells that retain the fundamental biologic attributes of antibody production and secretion. MM afflicts more than 30,000 Americans each year and its rate has been increasing consistently. In the early 21st century, with the introduction of proteasome inhibitors (PIs), such as bortezomib and carfilzomib, and immunomodulatory drugs (IMiDs), such as lenalidomide and pomalidomide, the average life expectancy of MM patients improved markedly. Beginning in 2015, daratumumab emerged as an important treatment option for this population. However, MM remains incurable, and drug resistance to all the above agents is inevitable. MM that becomes resistant to bortezomib, carfilzomib, lenalidomide, pomalidomide, and daratumumab is referred to as “penta-refractory,” with a median survival of only approximately nine months. Throughout the course of the disease, patients are debilitated by bone tumors, spontaneous fractures, kidney failure, immunosuppression, and eventually this course becomes fatal when the disease becomes treatment refractory. New early- and late-line therapies for myeloma, including therapies that retain their activity in advanced MM patients who have developed IMiD, PO, and daratumumab resistance are urgently needed.

### SUMMARY

In a first example (“Example 1”), provided herein is a method of treating multiple myeloma in a subject, comprising administering to the subject: i) a therapeutically effective amount of at least one protein translation inhibitor and a therapeutically effective amount of at least one immunomodulatory drug (IMiD); ii) a therapeutically effective amount of at least one protein translation inhibitor and a therapeutically effective amount of at least one bromodomain extra-terminal (BET) inhibitor; or iii) a therapeutically effective amount of at least one IMiD and a therapeutically effective amount of at least one BET inhibitor.

In a second example (“Example 2”), provided herein is at least one protein translation inhibitor and at least one immunomodulatory drug (IMiD), at least one protein translation inhibitor and at least one bromodomain extra-terminal (BET) inhibitor, or at least one IMiD and at least one BET inhibitor, for use in a method of treating multiple myeloma in a subject, the method comprising administering to the subject a therapeutically effective amount of the at least one protein translation inhibitor and a therapeutically effective amount of the at least one IMiD, administering to the subject a therapeutically effective amount of the at least one protein translation inhibitor and the at least one BET inhibitor, or administering to the subject a therapeutically effective amount of the at least one IMiD and the at least one BET inhibitor.

In another example (“Example 3”), further to Example 1 or Example 2, i) a first pharmaceutical composition or group of pharmaceutical compositions comprises the therapeutically effective amount of the at least one protein translator inhibitor, and a second pharmaceutical composition or group of pharmaceutical compositions comprises the at least one IMiD; ii) a first pharmaceutical composition or group of pharmaceutical compositions comprises the therapeutically effective amount of the at least one protein translator inhibitor, and a second pharmaceutical composition or group of pharmaceutical compositions comprises the at least one BET inhibitor; or iii) a first pharmaceutical composition or group of pharmaceutical compositions comprises the therapeutically effective amount of the at least one IMiD, and a second pharmaceutical composition or group of pharmaceutical compositions comprises the at least one BET inhibitor.

In another example (“Example 4”), further to Example 1 or Example 2, a single pharmaceutical composition comprises: i) the therapeutically effective amount of the at least one protein translation inhibitor and the therapeutically effective amount of the at least one IMiD; ii) the therapeutically effective amount of the at least one protein translation inhibitor and the therapeutically effective amount of the at least one BET inhibitor, or iii) the therapeutically effective amount of the at least one IMiD and the therapeutically effective amount of the at least one BET inhibitor.

In another example (“Example 5”), further to any one of Examples 1-4, the method further comprises administering to the subject a therapeutically effective amount of at least one additional agent that is: i) neither a protein translation inhibitor or an IMiD wherein a protein translation inhibitor and an IMiD are administered to the subject; ii) neither a protein translation inhibitor or a BET inhibitor wherein a protein translation inhibitor and a BET inhibitor are administered to the subject; or iii) neither an IMiD or a BET inhibitor wherein an IMiD and a BET inhibitor are administered to the subject.

In another example (“Example 6”), further to Example 5, the at least one additional agent that is neither a protein translation inhibitor or an IMiD is selected from the group of: dexamethasone, prednisone, prednisolone, methylprednisolone, hydrocortisone, bortezomib, carfilzomib, marizomib, ixazomib, oprozomib, bendamustine, carmustine, cyclophosphamide, melphalan, melphalan hydrochloride, afuresertib, ibrutinib, dinacicib, panobinostat, rocilinostat, vorinostat, siltuximab, filanesib, daratumumab, elotuzumab, indatuximab, SAR650984, doxorubicin hydrochloride, panobinostat, plerixafor, and selinexor.

In another example (“Example 7”), further to any one of Examples 1-6, the at least one protein translation inhibitor is selected from the group of: omacetaxine mepesuccinate, anisomycin, lactimidomycin, cycloheximide, verrucarin A, cephaeline, emetine, bouvardin, puromycin, didemnins, and bruceantin; the at least on IMiD is selected from the group of: lenalidomide, pomalidomide, thalidomide, and iberdomide; and the at least one BET inhibitor is selected from the group of: JQ1, ABBV-075, FT-1101, GSK525762 (I-BET762), INCB057643, ZEN003694, OTX015 (MK-8628), GSK2820151 (I-BET151), CC-90010, CPI-0610, PLX51107, ABBV-744, BI 894999, BMS-986158, GS-5829, INCB054329, and RO6870810 (TEN-010).

In another example (“Example 8”), further to any one of Examples 1-7, the at least one protein translation inhibitor comprises omacetaxine mepesuccinate, and the therapeutically effective amount of omacetaxine mepesuccinate inhibitor is about 1.25 mg/m².

In another example (“Example 9”), further to any one of Examples 1-7, the at least one protein translation inhibitor comprises lenalidomide, and the therapeutically effective amount of lenalidomide is about 2.5 mg to about 25 mg.

In another example (“Example 10”), further to any one of Examples 1-7, the at least one IMiD comprises pomalidomide, and the therapeutically effective amount of pomalidomide is about 1 mg to about 4 mg.

In another example (“Example 11”), further to any one of Examples 1-7, the at least one IMiD comprises thalidomide, and the therapeutically effective amount of thalidomide is about 200 mg.

In another example (“Example 12”), further to any one of Examples 1-7, the at least one IMiD comprises iberdomide, and the therapeutically effective amount of iberdomide is about 0.3 mg to about 1.2 mg.

In another example (“Example 13”), further to any one of Examples 1-12, the subject is resistant to one or more multiple myeloma therapeutic agents.

In another example (“Example 14”), further to Example 13, the subject is resistant to one or more multiple myeloma therapeutic agents selected from: an IMiD, a proteasome inhibitor, and an antibody.

In a fifteenth example (“Example 15”), provided herein is a method of treating multiple myeloma in a subject, comprising administering to the subject a therapeutically effective amount of at least one protein translation inhibitor.

In another example (“Example 16”), further to Example 15, the at least one protein translation inhibitor is selected from the group of: omacetaxine mepesuccinate, anisomycin, and lactimidomycin.

In another example (“Example 17”), further to Example 15 or 16, the at least one protein translation inhibitor does not comprise omacetaxine mepesuccinate, or is not omacetaxine mepesuccinate.

In another example (“Example 18”), further to Example 15 or 16, wherein the at least one protein translation inhibitor comprises omacetaxine mepesuccinate, and the therapeutically effective amount of omacetaxine mepesuccinate inhibitor is about 1.25 mg/m².

In another example (“Example 19”), further to any one of Examples 15-18, the method includes administering to the subject a therapeutically effective amount of at least one additional agent that is not a protein translation inhibitor.

In another example (“Example 20”), further to Example 18, wherein the at least one agent is selected from the group of: lenalidomide, pomalidomide, thalidomide, iberdomide, dexamethasone, prednisone, prednisolone, methylprednisolone, hydrocortisone, bortezomib, carfilzomib, marizomib, ixazomib, oprozomib, bendamustine, carmustine, cyclophosphamide, melphalan, melphalan hydrochloride, afuresertib, ibrutinib, dinaciclib, panobinostat, rocilinostat, vorinostat, siltuximab, filanesib, daratumumab, elotuzumab, indatuximab, SAR650984, doxorubicin hydrochloride, panobinostat, plerixafor, and selinexor.

In another example (“Example 21”), further to any one of examples 15-20, the subject is resistant to one or more anti-multiple myeloma agents.

In another example (“Example 22”), further to Example 21, the subject is resistant to one or more anti-multiple myeloma agents selected from: an IMiD, a proteasome inhibitor, and an antibody.

### BRIEF DESCRIPTION OF THE DRAWINGS

The accompanying drawings are incorporated into and form a part of the specification to illustrate several examples of the present disclosure. The drawings simply illustrate examples of how the disclosure can be made and used and are not to be construed as limiting the disclosure to the illustrated and described examples. Further features and advantages will become apparent from the following, more detailed, description of the various aspects, embodiments, and configurations of the disclosure, as illustrated by the drawings referenced below.

FIG. 1A is a line graph depicting cell proliferation assay results for five MM cell lines treated with increasing doses of omacetaxine (Oma) for 96 h.

FIG. 1B illustrates co-staining with Annexin V and DAPI of the MM.1S cell line treated with 50 nM omacetaxine for 48 h.

FIG. 1C depicts the timecourse of the start of omacetaxine's induction of apoptosis in MM1.S cells.

FIG. 1D is a bar graph illustrating reduced myeloma cell line metabolism by omacetaxine as measured by ECAR (extracellular acidification rate) after 4 h incubations.

FIG. 1E depicts flow cytometry gating results after ex vivo treatment of primary MM cells from a patient with 50 nM omacetaxine for 48 h. Live cells were gated, followed sequentially by CD45dim−/CD19− and finally CD38+/CD138+.

FIG. 1F is a line graph depicting dose response curves for six different MM patient primary samples treated with increasing concentrations of omacetaxine for 48 h measured by a decline in viable MM cells via multicolor flow cytometry

FIG. 1G is a waterfall plot illustrating the ex vivo effect of 50 nM omacetaxine treatment for 48 h in 50 patient samples categorized based on their PI and IMiD resistance.

FIG. 2A depicts the timecourse of induction of apoptosis and cell death by omacetaxine in MM1.S cells as measured by Annexin V and DAPI staining by flow cytometry.

FIGS. 2B-2D depict results from a Seahorse glycolytic stress test of the H929 MM cell line (FIG. 2B), MM.1S MM cell line (FIG. 2C), and U266 MM cell line (FIG. 2D) incubated with omacetaxine (Oma, 50 nM) for 4 h.

FIG. 3A depicts translation levels in primary MM cells and normal marrow mononuclear cells (MNCs) from 17 patients measured using OP-Puro and flow cytometry.

FIG. 3B provides paired T-test of translation levels in primary MM cells compared to the normal MNCs.

FIG. 3C depicts the dose-dependent response curves of translation levels with increasing omacetaxine in the H929 MM cell line and in two primary MM cell samples.

FIG. 3D illustrates omacetaxine's (Oma, 50 nM) ability to inhibit translation in primary MM cells after 2.5 h.

FIG. 3E illustrates that the ex vivo sensitivity of primary MM cells to omacetaxine correlates with baseline translation levels. High translation was defined as 2.5-fold or greater translation compared to MNCs.

FIG. 3F illustrates MCL1 antigen density on primary MM cells compared to MNCs.

FIG. 3G illustrates translation levels of six MM cell lines at baseline and after 2.5 h of 50 nM omacetaxine treatment.

FIG. 3H illustrates MCL1 antigen density on a primary myeloma samples treated with 50 nM omacetaxine for 6 h.

FIG. 4A presents a comparison of baseline translation levels between newly diagnosed MM patient samples and relapse MM patient samples.

FIG. 4B is a bar graph illustrating inhibition of protein translation levels of MM cell lines by 2.5 h of 50 nM omacetaxine treatment.

FIG. 4C is a bar graph illustrating that Boitezomib treatment did not change translation levels over 6 h, 24 h, and 48 h in MM cell lines.

FIG. 4D illustrates that plasma cells have similar baseline translation levels compared to primary myeloma cells.

FIG. 4E illustrates healthy donor plasma cell sensitivity to omacetaxine (Oma, 50 nM) after 48 h incubation.

FIG. 5A is a bar graph showing that Bcl-2 does not decrease with omacetaxine treatment in myeloma cells. MCL1 mean fluorescence intensity (MFI) on myeloma cell lines following 6 h of 50 nM omacetaxine (Oma) treatment measured by intracellular flow cytometry.

FIG. 5B provides dose response curves of primary myeloma samples to the MCL1 inhibitor S63.

FIG. 5C is a bar graph summarizing the results from a BH3 priming assay by flow cytometry in MCL1-dependent H929 cells displaying NOXA induction of cytochrome c loss.

FIG. 5D summarizes BH3 priming results for MCL1 and Bcl-2 in primary myeloma patient samples. Green indicates primed.

FIG. 5E shows that MCL1 inhibitor sensitivity correlates with BH3 priming results in primary myeloma.

FIG. 5F shows omacetaxine sensitivity compared to Mcl-1 priming in nine primary myeloma samples.

FIG. 6A is a bar graph illustrating myeloma cell line viability after 96 h treatment with 20 μM lenalidomide (Len) and 15 nM omacetaxine (Oma) as single agents or in combination.

FIG. 6B depicts a ZIP synergy plot of Len and omacetaxine combinations matrix in MM.1S cells after 96 h treatment.

FIG. 6C is a bar graph illustrating myeloma cell line viability after 96 h treatment with 20 μM pomalidomide (Pom) and 15 nM omacetaxine as single agents or in combination.

FIG. 6D depicts a ZIP synergy plot of Pom and omacetaxine combinations matrix in MM.1S cells after 96 h treatment.

FIG. 6E is a line graph showing that combination treatment with Pom and omacetaxine was synergistic and restored Pom sensitivity in an MM patient sample.

FIG. 6F depicts a ZIP synergy plot of Pom and omacetaxine combinations matrix in patient sample HTB-576, δ score=22.7.

FIGS. 7A-7B illustrate that the combination of omacetaxine and bortezomib (Bor) exhibited no significant enhanced activity over single agent omacetaxine in U266, MM.1S and H929 cells.

FIGS. 7C-7D illustrate that the combination of omacetaxine with dexamethasone displayed decreased activity over single agent omacetaxine in L363 and U266 cells, but was synergistic in MM.1S cells.

FIG. 8A shows that the translation inhibitor anisomycin (Ani) and Pom are synergistic in MM.1S and H929 cells.

FIG. 8B shows that the IMiD CC220 and Oma are synergistic in H929 cells.

FIG. 8C shows that the translation inhibitor cycloheximide (Cyclo) and Pom are synergistic in MM.1S and H929 cells.

FIG. 8D shows that the translation inhibitor lactimidomycin (Lac) and Pom are synergistic in MM.1S and H929 cells.

FIG. 9A is a schematic showing the myeloma luciferase xenograft model. NSG mice were injected with 500,000 MM.1S cells, and the engraftment was confirmed after 30 days via IVIS bioluminescence imaging. Mice were injected with vehicle or with 1 mg/kg, 2 mg/kg, or 3 mg/kg Oma. After 1 h, 500 μg puromycin was given IP, and the proteins were labeled for 1.5 h before the bone marrow was harvested and stained.

FIG. 9B is a line graph illustrating the percentage of translation inhibition with increasing doses of Oma.

FIG. 9C is a schematic illustrating the design of the combo survival study. NSG mice were injected with 500,000 MM.1S cells, and the engraftment was confirmed after 30 days via IVIS bioluminescence imaging. Mice were injected with vehicle, 1 mg/kg Oma, 8 mg/kg Pom, or the combination of 1 mg/kg Oma and 8 mg/kg Pom. OP Puro, O-Propargyl-puromycin.

FIG. 9D depicts luciferase imaging of MM.1S xenografts that were either untreated, or treated with omacetaxine, pomalidomide, or the combination of both drugs.

FIG. 9E depicts Kaplan-Meier survival of combination studied in the in vivo MM model.

FIG. 9F is a table providing a comparison of mouse survival between the noted treatment protocols, providing the hazard ratio (HR) and corresponding p-values for each comparison.

FIG. 10A depicts the timecourse of induction of apoptosis and cell death by pomalidomide in MM1.S cells as measured by annexin V and DAPI staining by flow cytometry.

FIGS. 10B-10D are bar graphs summarizing the Seahorse glycolytic capacity of H929 cells (FIG. 10B), MM1.S (FIG. 10C) or U266 (FIG. 10D) incubated with Oma for 4 h, Pom for 24 h, or combination. ECAR—extracellular acidification rate.

FIG. 11A depicts a 2D Scores plot of proteomic results from MM.1S cells treated for 24 h Pom, 4 h with Oma, or with combination therapy versus untreated cells.

FIG. 11B is a heat map of the top 25 most differentially affected proteins following omacetaxine, pomalidomide and combination treatment, generated utilizing the Ward clustering algorithm and Euclidean distance measure.

FIGS. 11C-11E depict proteomic spectral read value comparison of IGL1 (FIG. 12B), JCHAIN (FIG. 12C), and IRF4 (FIG. 12D) across treatment groups.

FIG. 11F depicts spectral counts of two of the proteins most uniquely decreased by the combination treatment FUBP1 and SRF10.

FIG. 11G depicts a volcano plot of proteins from untreated MM.1S cells vs MM.1S cells after 4 h Oma treatment. Pink dots have greater than 2-fold change and p<0.05.

FIG. 12A illustrates a model of omacetaxine and IMiD combination therapy to cooperatively downregulate the IRF4/c-MYC axis in multiple myeloma cells. Green arrows indicate IMiD effect, red arrows omacetaxine effect and purple arrows combination effect.

FIG. 12B shows omacetaxine treatment of MM cell lines for 24 h decreases the levels of the c-MYC protein as measured by quantitative flow cytometry to determine the antigen density.

FIG. 13A shows results of in vitro combination therapy with the BET inhibitor JQ1 (50 nM) and omacetaxine (40 nM) in the MM.1S cell line for 96 h including ZIP synergy analysis. Data represent means±SD, comparisons by ANOVA, *p<0.05, **p<0.01, ***p<0.001.

FIG. 13B shows results of in vitro combination therapy with the BET inhibitor JQ1 (100 nM) and pomalidomide (1.25 μM) in the cell line U266 for 96 h including ZIP synergy analysis. Data represent means±SD, comparisons by ANOVA, *p<0.05, **p<0.01, ***p<0.001.

FIG. 13C shows ZIP synergy results of in vitro combination therapy with the BET inhibitor ABBV-075 and omacetaxine (left panel) or pomalidomide (right panel) in the MM.1S cell line.

FIG. 13D shows ZIP synergy results of in vitro combination therapy with the BET inhibitor OTX015 and omacetaxine in the MM.1S cell line.

### DETAILED DESCRIPTION

In the following sections, various compositions and methods are described in order to detail various embodiments. Practicing the various embodiments does not require the employment of all of the specific details outlined herein, but rather concentrations, times, and other specific details may be modified. In some cases, well known methods or components have not been included in the description.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, and/or (4) to slow the progression of the condition or one or more of the biological manifestations of the condition. Treatment of multiple myeloma can result in an increase in average survival time of a population of treated subjects relative to a control population

As used herein, “therapeutically effective amount” in reference to an agent means an amount of the agent sufficient to treat the subject's condition but low enough to avoid serious side effects at a reasonable benefit/risk ratio within the scope of sound medical judgment. The amount of the agent sufficient to treat the subject's condition may be lower when the agent is included in a synergistic combination with one or more additional agents than it would be as a monotherapy. The safe and effective amount of an agent will vary with the particular agent chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the nature of co-therapies included in a combination therapy; the desired therapeutic effect; and like factors, but can nevertheless be determined by the skilled artisan.

For any compound, agent, or composition, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LDs (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

A “subject” means any individual having, having symptoms of, or at risk for multiple myeloma. A subject may be human or non-human, and may include, for example, animals or species used as “model systems” for research purposes, such as a mouse model described herein. Subject may refer to an individual having a first diagnosis of multiple myeloma, or an individual with relapsed and/or refractory multiple myeloma. In certain embodiments, the subject is a human patient diagnosed with multiple myeloma.

As used herein, a “pharmaceutical composition” is a formulation containing a compound or agent (e.g., monoclonal antibody) in a form suitable for administration to a subject. Compounds and agents disclosed herein each can be formulated individually or in any combination into one or more pharmaceutical compositions. Accordingly, one or more administration routes can be properly elected based on the dosage form of each pharmaceutical composition. Alternatively, a compound or agent disclosed herein and one or more other therapeutic agents described herein can be formulated as one pharmaceutical composition.

Embodiments of the present disclosure provide multiple myeloma (MM) combination therapies based on protein translation inhibitors, immunomodulators, and bromodomain extra-terminal (BET) inhibitors. Methods are provided for treating multiple myeloma in a subject, including administering a therapeutically effective amount of at least one protein translation inhibitor and a therapeutically effective amount of at least one immunomodulatory drug (IMiD), administering a therapeutically effective amount of at least one protein translation inhibitor and a therapeutically effective amount of at least one BET inhibitor, and/or administering a therapeutically effective amount of at least one IMiD and a therapeutically effective amount of at least one BET inhibitor.

While combination therapies are more efficacious in myeloma treatment than single agents, it can generally not be predicted which combination of anti-myeloma agents will work best in a particular subject. Because of this, and particularly in cases of relapsed/refractory multiple myeloma, a ‘shotgun’ approach is often taken, trying various combinations of anti-myeloma agents until a particular combination is found to be effective. Currently available drugs for use in MM include proteasome inhibitors (PIs), such as bortezomib and carfilzomib, immunomodulatory drugs (IMiDs) such as lenalidomide and pomalidomide, and the monoclonal antibodies daratumumab and elotuzumab. Combination therapies commonly include an IMiD, a proteasome inhibitor, and corticosteroids. However, MM remains incurable, and drug resistance to bortezomib, carfilzomib, lenalidomide, pomalidomide, and daratumumab (termed “penta-refractory”) is inevitable.

Drugs affecting protein translation in MM cells—protein translation inhibitors—offer a class of drugs for use in the treatment of MM. Omacetaxine mepesuccinate (also known as homoharringtonine; herein referred to as omacetaxine or Oma), an antileukemic drug first isolated from the Chinese evergreen Cephalotaxus harringonia, was described to inhibit growth of MM cells in vitro, and to treat two refractory MM patients in combination with vincristine and dexamethasone. Omacetaxine is FDA-approved for chronic myeloid leukemia (CML) and acts via a unique mechanism among anti-cancer drugs by binding the A-site cleft of ribosomes and blocking protein synthesis. Described herein is evidence that protein translation inhibitors as a class can be used in the treatment of MM. Data presented in the Examples and Figures clearly indicate that in addition to omacetaxine, protein translation inhibitors anisomycin, cycloheximide, and lactimidomycin have consistent anti-myeloma efficacy against myeloma cells.

In certain embodiments, provided herein are methods for treating multiple myeloma in a subject, including administering to the subject a therapeutically effective amount of at least one protein translation inhibitor. The at least one protein translation inhibitor can include, for example, omacetaxine, anisomycin, and lactimidomycin. In some embodiments, the at least one protein translation inhibitor does not include omacetaxine, or is not omacetaxine. Use of protein translation inhibitors can be useful in subjects who are resistant to one or more anti-myeloma agents, such as, for example IMiDs, proteasome inhibitors, monoclonal antibodies, or a combination of thereof.

This disclosure is based on, at least in part, the discovery of the anti-MM synergism of various protein translation inhibitors with IMiDs, of protein translation inhibitors with BET inhibitors, and of IMiDs with BET inhibitors. This surprising synergism was identified with combinations of protein translation inhibitors omacetaxine, anisomycin, lactimidomycin, and cycloheximide. Even in MM cells resistant to IMiDs, the combination of a protein translation inhibitor with an IMiD had a significant anti-MM effect, demonstrating a resensitization of the MM cells to the IMiD. This synergistic effect is due to the protein translation inhibitors and the IMiDs each affecting the same key proteins (see Example 4 herein). This synergistic effect did not occur when a protein translation inhibitor was combined with a proteasome inhibitor (see Example 3 herein), evidencing a unique effect between protein translation inhibitors and IMiDs.

It was further tested whether either protein translation inhibitors or IMiDs would demonstrate similar synergism with members of the BET inhibitor drug class. Synergism was observed in both cases.

Certain embodiments provide methods for treating multiple myeloma in subject, including administering to the subject a therapeutically effective amount of at least one protein translation inhibitor and a therapeutically effective amount of at least one IMiD, administering a therapeutically effective amount of at least one protein translation inhibitor and a therapeutically effective amount of at least one BET inhibitor, and/or administering a therapeutically effective amount of at least one IMiD and a therapeutically effective amount of at least one BET inhibitor.

In some embodiments, the at least one protein translation inhibitor includes, but is not limited to, omacetaxine, anisomycin, lactimidomycin, cycloheximide, verrucarin A, cephaeline, emetine, bouvardin, puromycin, didemnins, bruceantin, and combinations of any two or more thereof. In some embodiments, the at least one protein translation inhibitor is omacetaxine.

In some embodiments, the at least one IMiD includes, but is not limited to, lenalidomide, pomalidomide, thalidomide, and iberdomide (CC-220), and combinations of any two or more thereof.

In some embodiments, the at least one BET inhibitor includes, but is not limited to, JQ1, ABBV-075, FT-1101, GSK525762 (I-BET762), INCB057643, ZEN003694, OTX015 (MK-8628), GSK2820151 (I-BET151), CC-90010, CPI-0610, PLX51107, ABBV-744, BI 894999, BMS-986158, GS-5829, INCB054329, and RO6870810 (TEN-010).

The at least one protein translation inhibitor and the at least one IMiD, the at least one protein translator and the at least one BET inhibitor, and/or the at least one IMiD and the at least one BET inhibitor can be administered in combination simultaneously or via separate administrations. When administered separately the translation inhibitor. IMiD, and/or BET inhibitor are each administered within a timespan sufficient to ensure a synergistic effect between the two agents. In some embodiments, the two administered agents are administered in combination simultaneously, or within, for example, 1 min, 5 min, 10 min, 15 min, 30 min, 1 h, 2 h, 4 h, 6 h, 8 h, 12 h, 16 h, 20 h, or 24 h of each other.

In some embodiments, a first pharmaceutical composition or group of pharmaceutical compositions includes the therapeutically effective amount of a first agent (e.g., at least one protein translation inhibitor), and a second pharmaceutical composition or group of pharmaceutical compositions includes the therapeutically effective amount of a second agent (e.g., at least one IMiD). In embodiments where two or more different protein translation inhibitors are to be administered, each of the two or more different protein translation inhibitors can be included in separate pharmaceutical compositions, or can be combined into one or more pharmaceutical compositions. In embodiments where two or more different IMiDs are to be administered, each of the two or more different IMiDs can be included in separate pharmaceutical compositions, or can be combined into one or more pharmaceutical compositions. In embodiments where two or more different BET inhibitors are to be administered, each of the two or more different BET inhibitors can be included in separate pharmaceutical compositions, or can be combined into one or more pharmaceutical compositions.

In some embodiments, at least one protein translation inhibitor and at least one IMiD are included in a single pharmaceutical composition. In other embodiments, at least one protein translation inhibitor and at least one BET inhibitor are included in a single pharmaceutical composition. In yet other embodiments, at least one IMiD and at least one BET inhibitor are included in a single pharmaceutical composition. Certain aspects of the present disclosure thus provide pharmaceutical compositions including one or more protein translation inhibitors and one or more IMiDs, one or more protein translation inhibitors and one or more BET inhibitors, or one or more IMiDs and one or more BET inhibitors. A single pharmaceutical composition including two synergistic agents as describe herein can be formulated for a chosen route of administration, and the quantity of active ingredient in a unit dose of the composition is a therapeutically effective amount, which can be varied according to the particular treatment involved.

In some embodiments, the protein translation inhibitor to be administered to the subject according to the methods of this disclosure may be approved by a regulatory body, such as omacetaxine (FDA-approved), for example, or undergoing clinical trials. Where the protein translation inhibitor is approved or undergoing clinical trials, the therapeutically effective amount of the protein translation inhibitor can be the approved dosage, or the dosage(s) being investigated by clinical trial. In other embodiments, the protein translation inhibitor may not be approved or undergoing investigation in a clinical trial. The therapeutically effective amount of such inhibitors can be determined by the skilled artisan.

In some embodiments, the therapeutically effective amount of protein translation inhibitor will be lower when used in a combination therapy with an IMiD or a BET inhibitor, as described herein, in comparison to monotherapy. Such lower therapeutically effective amounts could afford for lower toxicity of the therapeutic regimen.

In some embodiments, the protein translation inhibitor is administered via an approved administration route or an administration route being investigated by clinical trial. In other embodiments, the protein translation inhibitor is administered via a different administration route. Where a protein translation inhibitor is to be administered via an administration route not previously approved or investigated, the amount of the protein translation inhibitor may be modified to ensure a therapeutically effective amount of the inhibitor is being administered. Those skilled in the art will recognize that different administration routes may affect the dosage required to achieve the desired result, and can make such adjustments.

In some embodiments, the therapeutically effective amount of omacetaxine is about 1.25 mg/m², delivered subcutaneously, twice daily. As described above, lower therapeutically effective amounts may be useful in the claimed combination therapy. This can be achieved by lowering the dosage or administering the agent less frequently.

The IMiD to be administered to the subject according to the methods of this disclosure may be approved by a regulatory body, such as lenalidomide, pomalidomide, and thalidomide, for example, or undergoing clinical trials, such as iberdomide (CC-220). Where the IMiD is approved or undergoing clinical trials, the therapeutically effective amount of the MCD can be the approved dosage, or the dosage(s) being investigated by clinical trial. In other embodiments, the IMiD may not be approved or undergoing investigation in a clinical trial. The therapeutically effective amount of such IMiDs can be determined by the skilled artisan.

In some embodiments, the therapeutically effective amount of IMiD will be lower when used in a combination therapy with a IMiD, as described herein, in comparison to monotherapy. Such lower therapeutically effective amounts could afford for lower toxicity of the therapeutic regimen.

In some embodiments, the IMiD is administered via an approved administration route or an administration route being investigated by clinical trial. In other embodiments, the IMiD is administered via a different administration route. Where an IMiD is to be administered via an administration route not previously approved or investigated, the amount of the IMiD may be modified to ensure a therapeutically effective amount of the inhibitor is being administered. Those skilled in the art will recognize that different administration routes may affect the dosage required to achieve the desired result, and can make such adjustments.

In some embodiments, the therapeutically effective amount of lenalidomide is about 2.5 mg to about 25 mg. Lenalidomide may be administered orally, and is available in capsules of 2.5 mg, 5 mg, 10 mg, 15 mg, 20 mg, and 25 mg.

In some embodiments, the therapeutically effective amount of pomalidomide is about 1 mg to about 4 mg. Pomalidomide may be administered orally, and is available in capsules of 1 mg, 2 mg, 3 mg, and 4 mg.

In some embodiments, the therapeutically effective amount of thalidomide is about 200 mg. Thalidomide may be administered orally, and is available in capsules of 50 mg, 100 mg, 150 mg, and 200 mg.

In some embodiments, the therapeutically effective amount of iberdomide is about 0.3 mg to about 1.2 mg. Iberdomide may be administered orally.

As described above, lower therapeutically effective amounts of these IMiDs may be useful in the claimed combination therapy. This can be achieved by lowering the dosage or administering the agent less frequently.

The BET inhibitor to be administered to the subject according to the methods of this disclosure may be approved by a regulatory body or undergoing clinical trials, such as, for example, ABBV-075, FT-1101, GSK525762 (I-BET762), INCB057643, ZEN003694, OTX015 (MK-8628), GSK2820151 (I-BET151), CC-90010, CPI-0610, PLX51107, ABBV-744, BI 894999, BMS-986158, GS-5829, INCB054329, and RO6870810 (TEN-010). Where the BET inhibitor is approved or undergoing clinical trials, the therapeutically effective amount of the BET inhibitor can be the approved dosage, or the dosage(s) being investigated by clinical trial. In other embodiments, the BET inhibitor may not be approved or undergoing investigation in a clinical trial (e.g., JQ1). The therapeutically effective amount of such IMiDs can be determined by the skilled artisan.

In some embodiments, the therapeutically effective amount of IMiD will be lower when used in a combination therapy with a BET inhibitor, as described herein, in comparison to monotherapy. Such lower therapeutically effective amounts could afford for lower toxicity of the therapeutic regimen.

In some embodiments, the BET inhibitor is administered via an approved administration route or an administration route being investigated by clinical trial. In other embodiments, the BET inhibitor is administered via a different administration route. Where a BET inhibitor is to be administered via an administration route not previously approved or investigated, the amount of the BET inhibitor may be modified to ensure a therapeutically effective amount of the inhibitor is being administered. Those skilled in the art will recognize that different administration routes may affect the dosage required to achieve the desired result, and can make such adjustments.

In some embodiments, in addition to administration of a protein translation inhibitor and an IMiD, administration of a protein translation inhibitor and a BET inhibitor, and/or administration of an IMiD and a BET inhibitor, the methods for treating multiple myeloma provided herein further include administering at least one additional agent.

In some embodiments, the at least one additional agent includes or is a corticosteroid. The corticosteroid may be, for example, dexamethasone, prednisone, prednisolone, methylprednisolone, and hydrocortisone.

In some embodiments, the at least one additional agent includes or is an anti-multiple myeloma agent that is not of the same class as the administered agents (e.g., a protein translation inhibitor or an IMiD), such as, for example, proteasome inhibitors (e.g., bortezomib, carfilzomib, marizomib, ixazomib, oprozomib), alkylating agents (e.g., bendamustine, carmustine, cyclophosphamide, melphalan, melphalan hydrochloride), AKT inhibitors (e.g., afuresertib), BTK inhibitors (e.g., ibrutinib), CDK inhibitors (e.g., dinaciclib), histone deacetylase inhibitors (e.g., panobinostat, rocilinostat, vorinostat), IL-6 inhibitors (e.g., siltuximab), kinesin spindle protein inhibitors (e.g., filanesib), monoclonal antibodies (e.g., daratumumab, elotuzumab, SAR650984 (isatuximab), bispecific antibodies, chimeric antigen receptor T-cells, antibody-drug conjugates, phosphoinositide 3-kinase (PI3K) inhibitors, doxorubicin hydrochloride, melflufen, panobinostat, plerixafor, and selinexor. In some embodiments, the at least one additional agent includes or is a protein translation inhibitor, where the administered combination therapy includes an IMiD and a BET inhibitor. In other embodiments, the at least one additional agent includes or is an IMiD, where the administered combination therapy includes a protein translation inhibitor and a BET inhibitor. In yet other embodiments, the at least one additional agent includes or is a BET inhibitor, where the administered combination therapy includes a protein translation inhibitor and an IMiD.

In some embodiments, the methods disclosed herein can be used at any stage of disease. For example, the methods for treating multiple myeloma described herein can be used as a first-line treatment or as a late-line treatment. In certain embodiments, the methods described herein can be used to treat a subject with relapsed or relapsed/refractory MM. In some embodiments, the subject to be treated is resistant to one or more available anti-MM treatments, including resistance to one or more of lenalidomide, pomalidomide, thalidomide, iberdomide, bortezomib, carfilzomib, marizomib, ixazomib, oprozomib, bendamustine, carmustine, cyclophosphamide, melphalan, melphalan hydrochloride, afuresertib, ibrutinib, dinaciclib, panobinostat, rocilinostat, vorinostat, siltuximab, filanesib, daratumumab, elotuzumab, indatuximab, SAR650984, doxorubicin hydrochloride, panobinostat, plerixafor, and selinexor. In particular embodiments, the subject to be treated is resistant to bortezomib, carfilzomib, lenalidomide, pomalidomide, and daratumumab (i.e., is multi-drug refractory).

In another aspect, provided herein is a method for predicting whether a subject is likely to respond to treatment with a protein translation inhibitor. In certain embodiments, the protein translation rate in a MM cell or a group of MM cells is determined and compared to the translation rate in a control or group of control marrow mononuclear cells (MNCs). Where baseline protein translation in the MM cell or group of MM cells is about 2.5-fold higher than in the control MNCs, the subject from which the MM cell or group of cells was isolated is considered to be sensitive to treatment with a protein translation inhibitor. Where the baseline protein translation in the MM cell or group of MM cells is less than 2.5-fold higher than in the control MNCs, the subject from which the MM cell or group of cells was isolated is considered to be relatively resistant to treatment with a protein translation inhibitor. In particular embodiments, the methods for predicting whether a subject is likely to respond to treatment with a protein translation inhibitor predicts whether a subject is likely to respond to treatment with omacetaxine.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Example 1—Omacetaxine has Broad Anti-Myeloma Efficacy Against Myeloma Cells

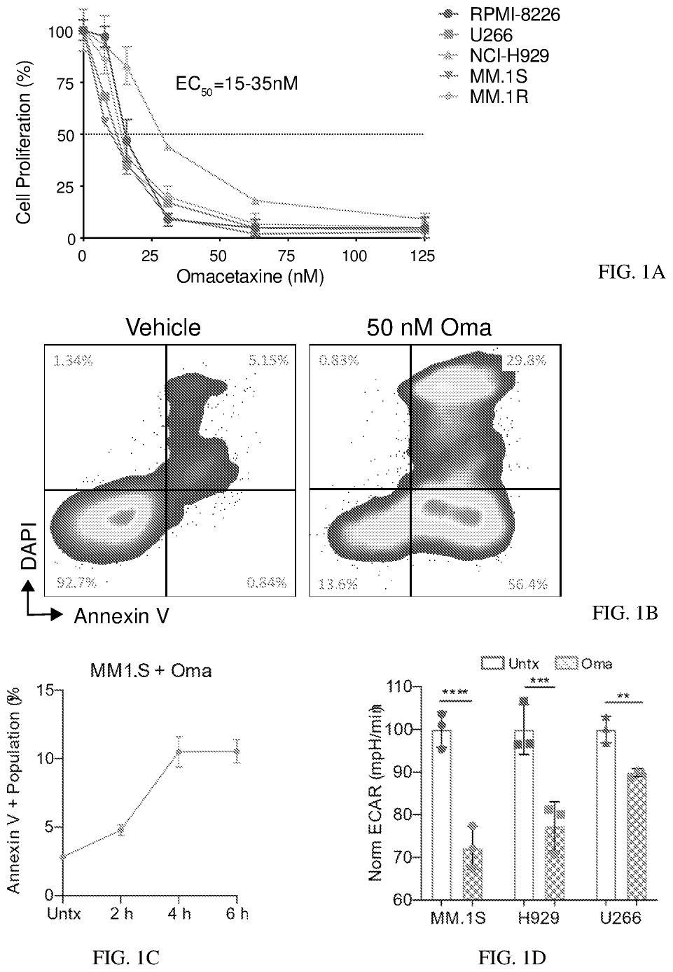

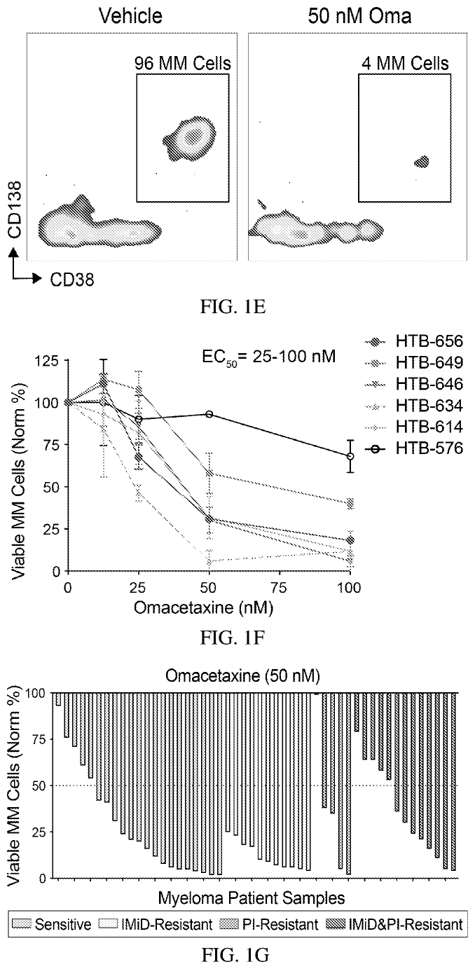

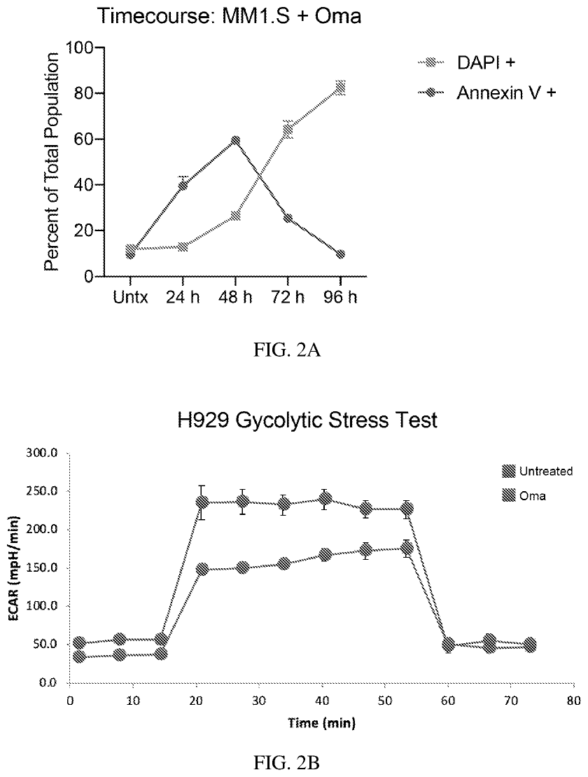

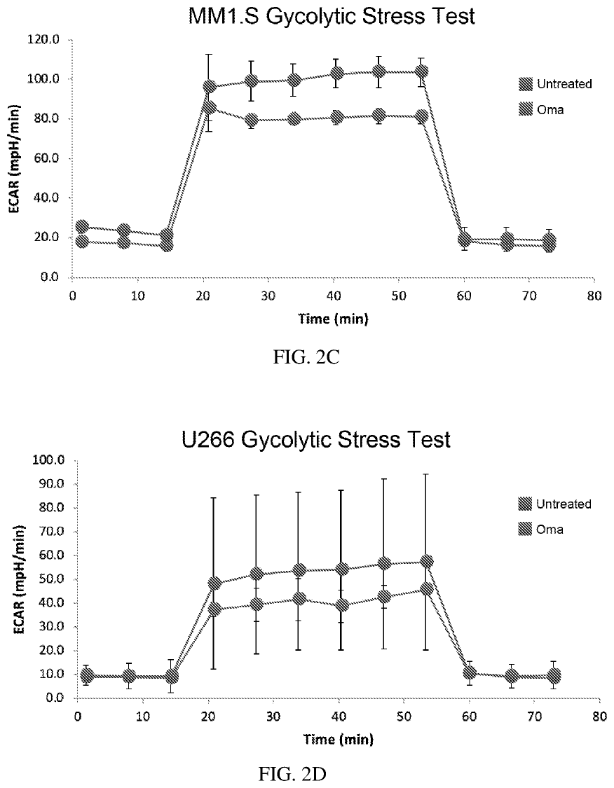

Omacetaxine was tested in MM cell lines in vitro and in MM patient bone marrow aspirates ex vivo. The reported cytotoxicity of omacetaxine was verified in cell culture using the U266, H929, MM1.S, MM1.R and RPMI-8226 MM cell lines. As illustrated in FIG. 1A, omacetaxine inhibited cell proliferation in the five different MM cell lines tested with an EC50 range of 15-35 nM after a 96 h incubation. In a timecourse study, omacetaxine-mediated induction of apoptosis started at 2 h, and cell death began after 24 h (FIGS. 1B, 1C, and 2A). In addition, omacetaxine treatment shut down metabolism in myeloma cell lines after 4 h, as measured by the Seahorse Assay (FIGS. 1D and 2B-2D). These data demonstrate the ability of omacetaxine to rapidly induce metabolic shut down, apoptosis, and cell death in all MM cell lines tested.

To evaluate clinical significance of the in vitro activity of omacetaxine in MM cell lines, omacetaxine activity was evaluated in primary cell samples from six different MM patients. The effect of omacetaxine in primary patient samples was tested by incubating marrow mononuclear cells (MNCs) from patient bone marrow aspirates for 48 h and measuring the number of surviving MM cells by multiparameter flow cytometry. As provided by FIGS. 1E and 1F, omacetaxine specifically induced apoptosis and decreases MM cell viability ex vivo with an EC50 range of 25-100 nM. Using a single concentration to screen additional primary cell samples from MM patients, ex vivo treatment with 50 nM omacetaxine for 48 h resulted in a >50% reduction in viable MM cells in 39/50 (78%) patient samples (FIG. 1G). Importantly, patients with relapsed/refractory myeloma who displayed resistance to PI and IMiDs did not show cross-resistance to Oma, which retained highly potent and specific anti-myeloma activity in single- and double-class refractory patients (FIG. 1G); omacetaxine retains anti-myeloma activity in multi-drug resistant patients.

### Example 2—Omacetaxine Inhibits Highly Active Protein Translation in Myeloma Cells

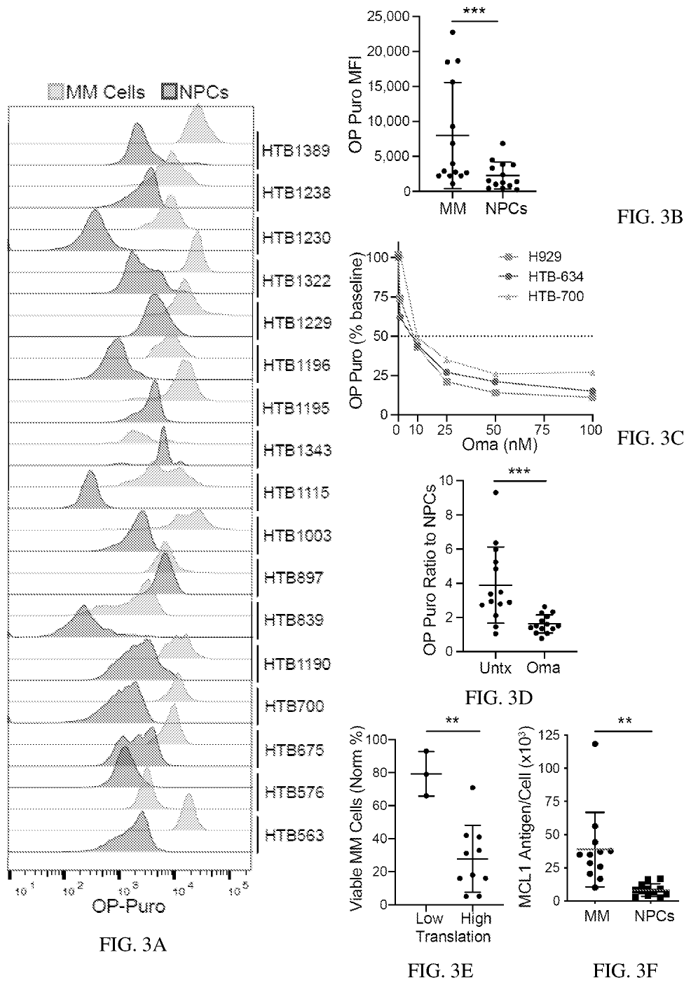

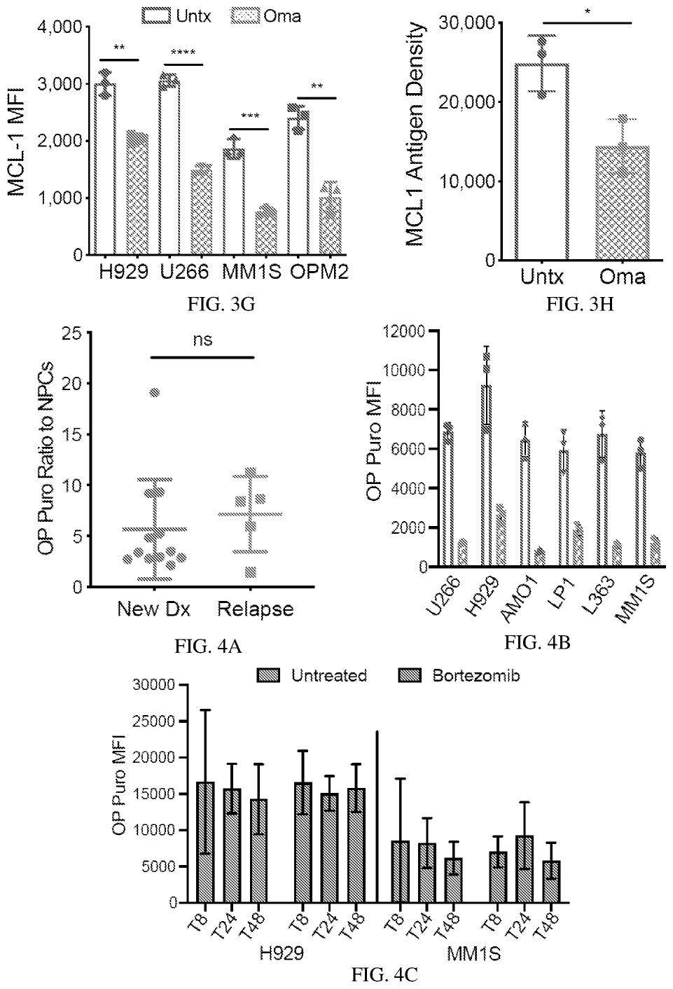

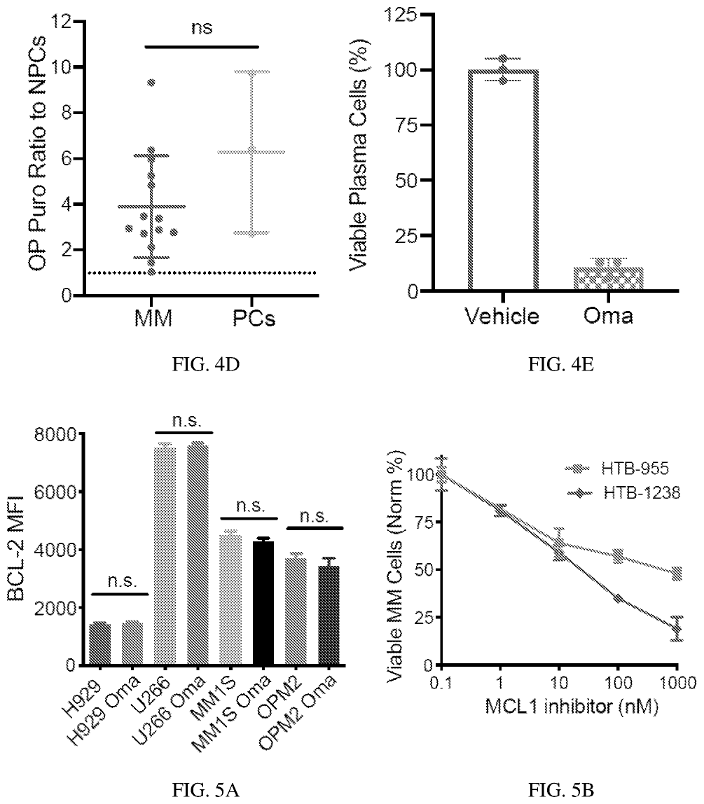

To examine whether myeloma cells depend on inherently high protein synthesis, the O-Propargyl-puromycin (OP-Puro) assay was used to measure the rate of amino-acid incorporation into translating ribosomes by flow cytometry. Based on the OP-puro assay, primary MM cells exhibited significantly higher levels of baseline protein translation compared to other bone marrow MNCs, with a mean 3.9-fold increase (n=14, P=<0.001) (FIGS. 3A and 3B). This high rate of protein translation was preserved in relapsed patient samples compared to newly diagnosed patient samples (FIG. 4A). Omacetaxine treatment for 2.5 h inhibited protein translation in a dose-dependent manner that correlated with its cytotoxicity in the H929 MM cell line and in primary patient MM cells with an EC₅₀ of 9 nM (FIG. 3C). The ability of omacetaxine to inhibit translation was observed across all myeloma cell lines tested, but did not occur with bortezomib treatment (FIGS. 4B and 4C). In 14 primary samples tested, translation was significantly reduced after 2.5 h of 50 nM omacetaxine treatment to near MNC levels (FIG. 3D). Using a cutoff of 2.5-fold higher baseline MM translation compared to MNCs, “High Translation” was associated with sensitivity to omacetaxine, and “Low Translation” was associated with relative resistance to omacetaxine (P=0.0018) (FIG. 3E). The high level of protein translation was also observed in bone marrow plasma cells from normal donors, and omacetaxine was also cytotoxic to these normal plasma cells (FIGS. 3D and 3E). These data demonstrate that protein translation inhibition is consistent across myeloma cells at concentrations that are also cytotoxic, and the translation rates serve as a biomarker for patients who are most likely to respond to omacetaxine treatment.

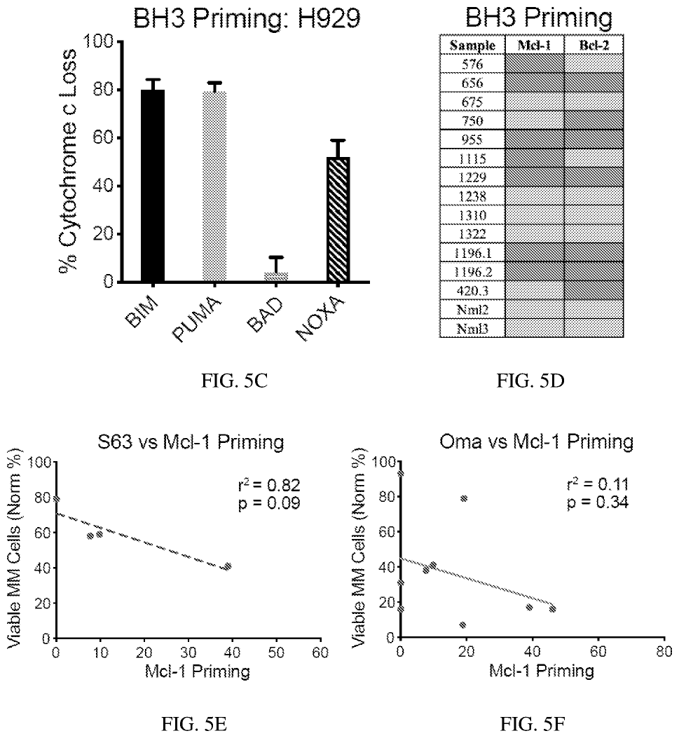

Omacetaxine-induced downregulation of the expression of proteins known to be important for MM cell survival, such as MCL1 or c-MYC, was investigated. By quantitative flow cytometry, MCL1 was confirmed to be overexpressed in MM cells and omacetaxine rapidly downregulated MCL1 but not BCL2 expression in myeloma cells (FIGS. 3F-3H and 5A). By comparison, the MCL1 inhibitor S63 was less cytotoxic than the broader acting omacetaxine (FIG. 5B). To determine whether MCL1 dependence may predict ex vivo response to omacetaxine, BH3 profiling for BCL2 family member priming was established, as demonstrated by FIG. 5C, with H929 cells known to be MCL1-primed. In a subset of the patient sample cohort, omacetaxine exhibited potent cytotoxicity against both MM cells that were primed or not primed for MCL1-mediated apoptosis (FIG. 5D). Whereas S63 cytotoxicity correlated well with MCL1 priming, omacetaxine demonstrated potent anti-myeloma activity in samples that were not MCL1 primed (FIGS. 5E and 5F). Thus, downregulation of MCL1, though important in myeloma cell survival, did not appear to fully explain the spectrum of omacetaxine activity against the disease.

### Example 3—Protein Translation Inhibitors Ae Synergistic with Immunomodulatory Drugs

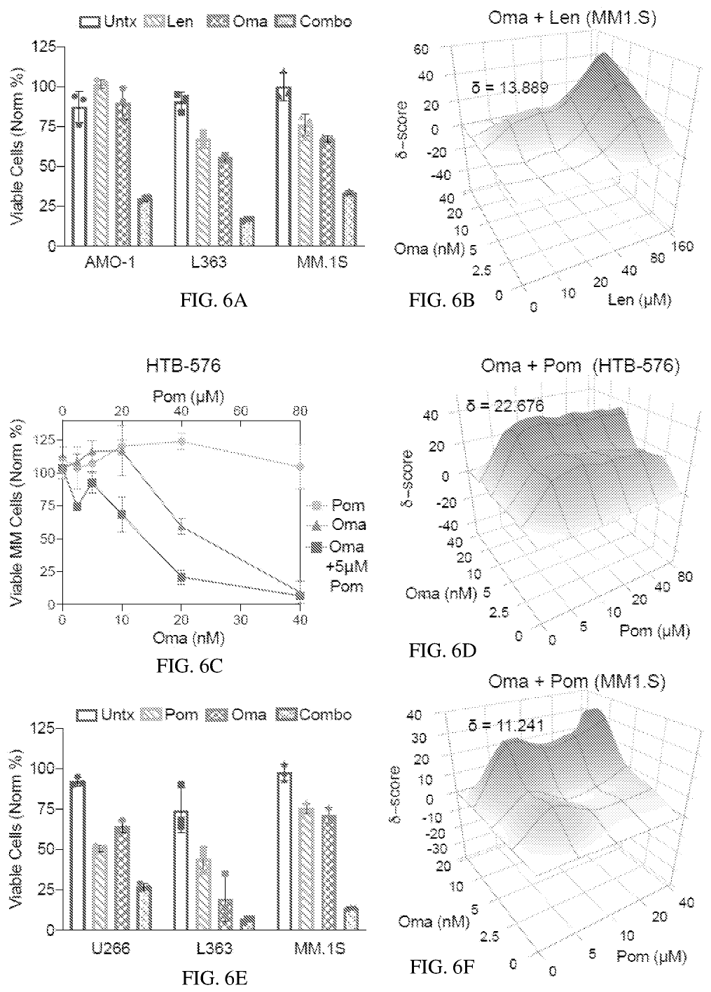

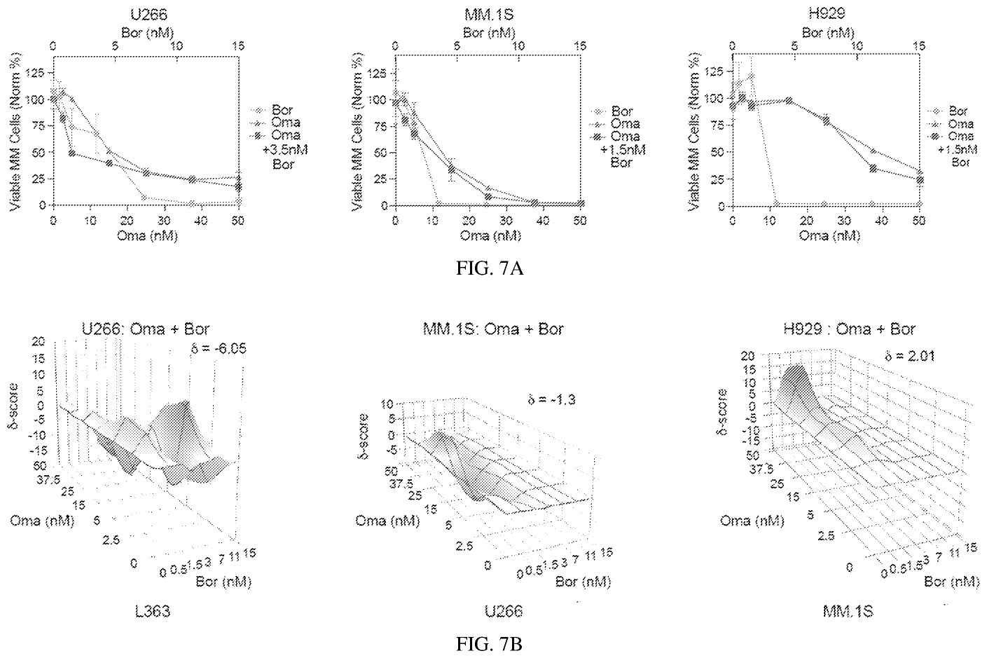

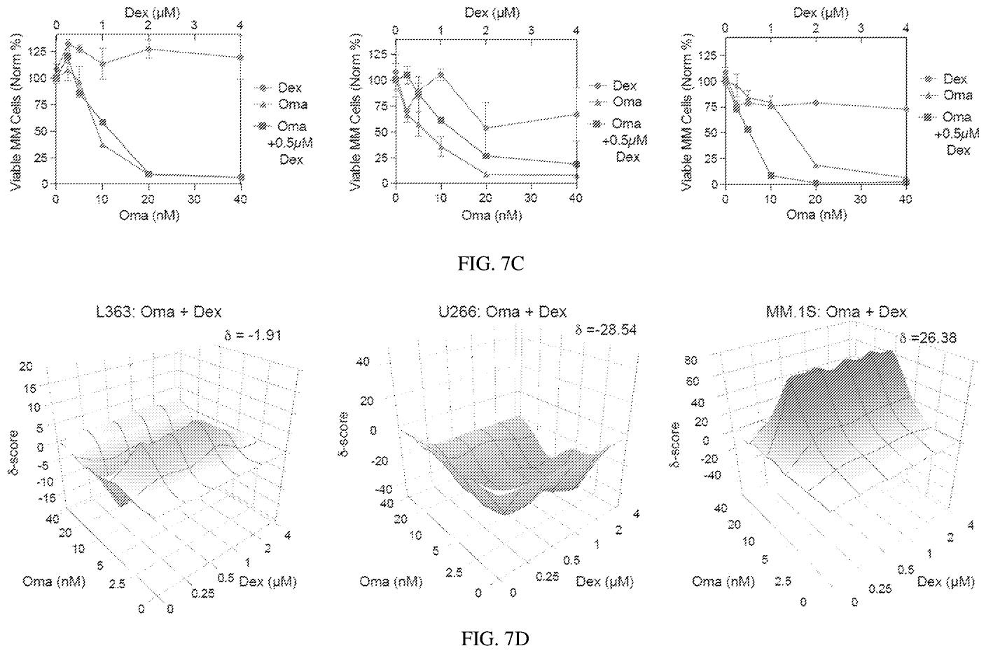

In this example, possible candidates for use in combination with omacetaxine were examined, focusing on those that are currently clinically available. Omacetaxine was applies in combination with various anti-myeloma agents to MM cell lines Amo-1, L363, and U266 in two-drug combination matrices, with cell viability being measured after 96 h incubation. Omacetaxine combined with lenalidomide (Len) or with pomalidomide (Pom) stood out as synergistic in all cell lines tested (FIGS. 6A and 6C). The 6-score for synergy of omacetaxine with Len and Pom were 13.89 and 11.24, respectively, with a broad range of concentrations showing a productive interaction (FIGS. 6B and 6D). Notably, in an IMiD-resistant relapsed patient sample, the combination of omacetaxine and Pom was even more synergistic and re-sensitized the MM cells to the IMiD (22.68 δ-score; FIGS. 6E and 6F). In contrast, omacetaxine combined with the PI bortezomib showed a lack of synergy when screened in the same manner in three MM cell lines (FIG. 7A). Omacetaxine combined with dexamethasone was synergistic in only ⅓ MM cell lines tested (FIG. 7B). When combined with omacetaxine, IMiDs displayed unique and consistent synergy that was not observed with other anti-myeloma drugs tested.

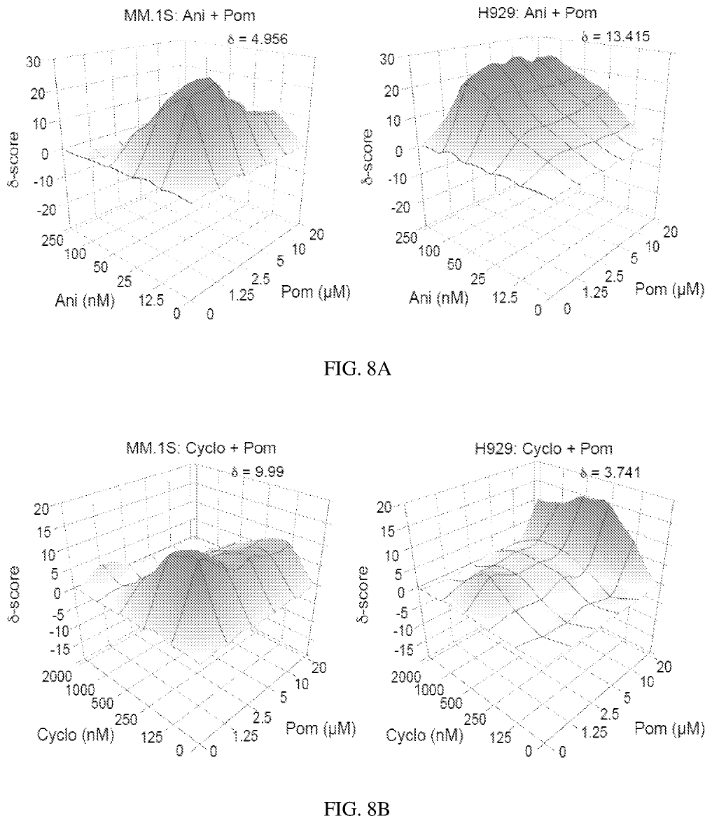

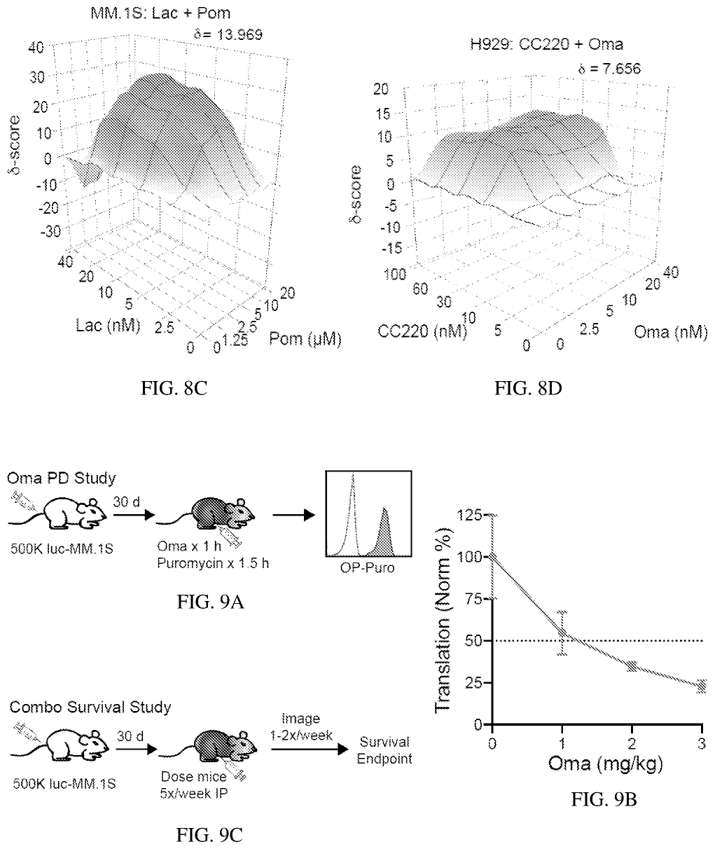

To assess whether the combination effects of omacetaxine with Len or Pom were broadly applicable across the translational inhibitor and IMiD classes, additional agents in each class were tested for synergistic anti-myeloma cytotoxicity. Omacetaxine was also synergistic with the new IMiD-derivative CC-220. Protein translation inhibitors anisomycin, cycloheximide, and lactimidomycin were also synergistic with pomalidomide (FIGS. 8A, 8C, and 8D). These results demonstrate that the synergy between these agents represents a broadly applicable class effect. Since pomalidomide is approved in relapsed/refractory myeloma, the setting most attractive for the application of omacetaxine, the omacetaxine/pomalidomide combination was studied further.

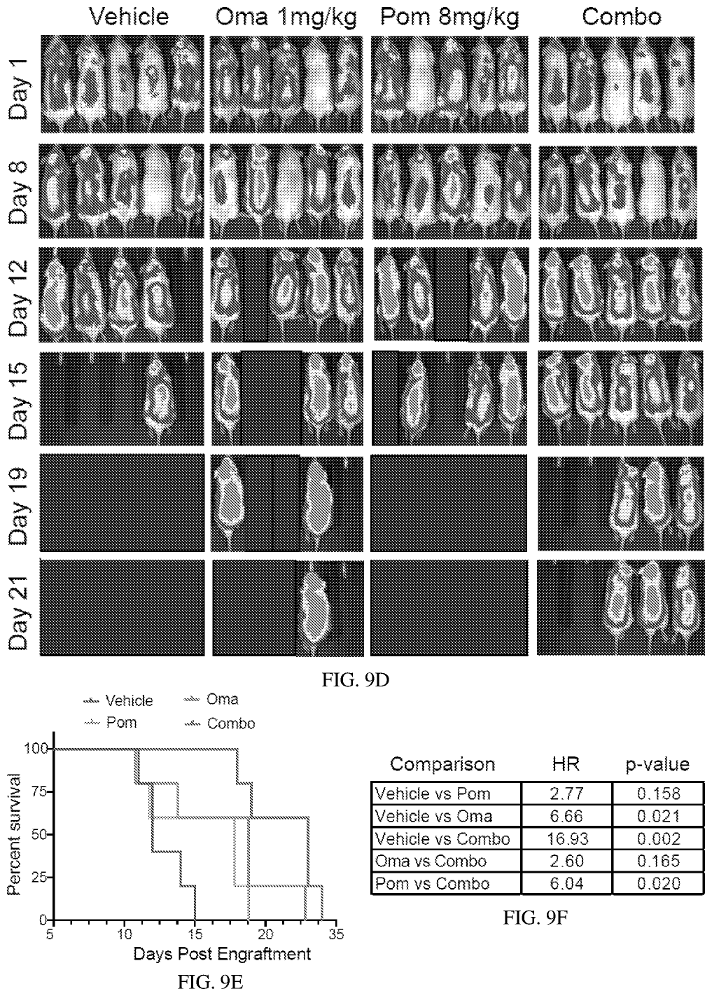

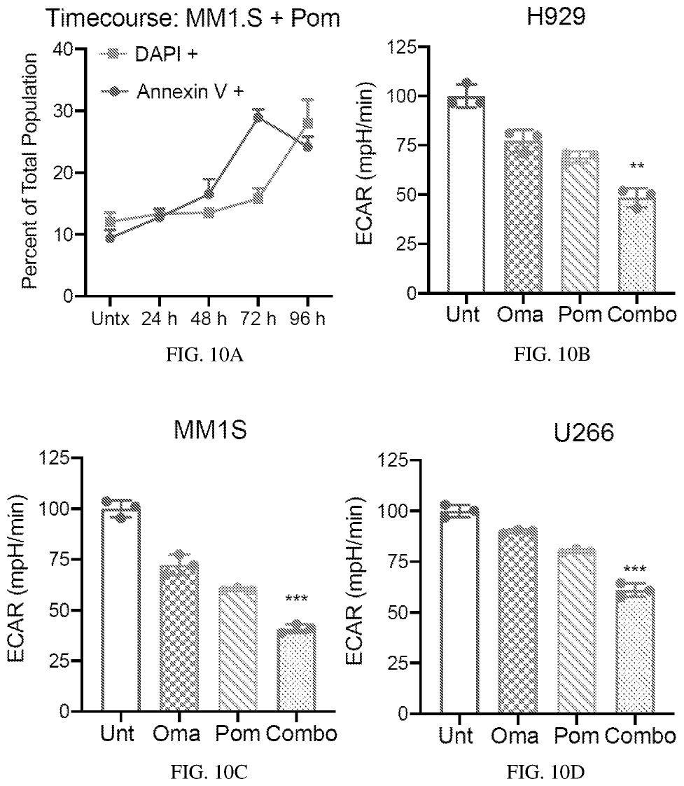

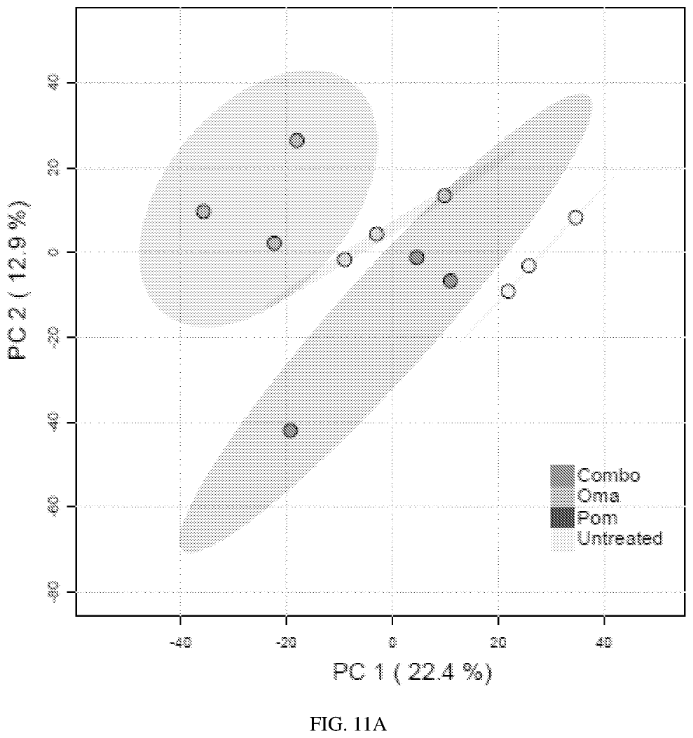

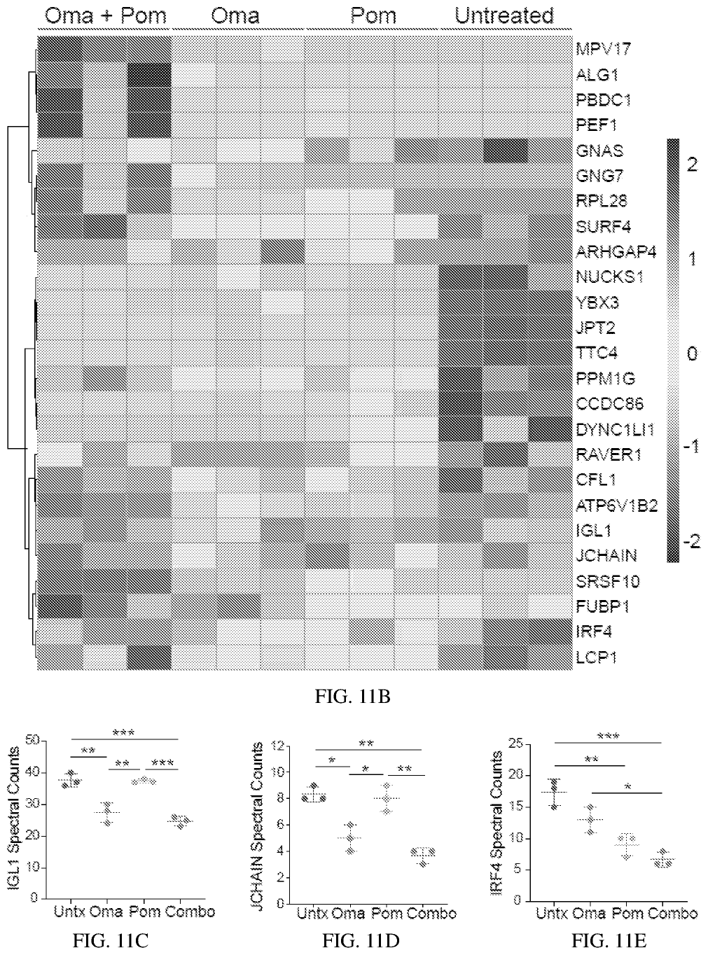

To extend the findings to an in vivo model, the effects of omacetaxine were examined using a firefly luciferase-expressing myeloma cell line, MM1.S (luc-MM1.S) to generate xenografts in NSG mice. The pharmacodynamics of omacetaxine were studied in this model. Mice were injected with luc-MM1.S cells and allowed to establish disease, followed by treatment with 1, 2, or 3 mg/kg omacetaxine, or with the vehicle control by intraperitoneal (IP) injection, followed by puromycin injection one hour later, and sacrificed 1.5 hours later, with bone marrow harvest and OP-puro incorporation by flow cytometry (FIG. 9A). A dose-dependent decrease in the protein translation rate in the luc-MM1.S cells was observed, with the greatest effect occurring at 3 mg/kg (FIG. 9B). These results were used to design a study to determine the benefit of combining omacetaxine with pomalidomide treatment in vivo

The combination treatment with omacetaxine and pomalidomide was tested in vivo. To evaluate for benefit of the drug combination, lower dose levels of omacetaxine (1 mg/kg) and Pom (8 mg/kg) were used in the in vivo experiment. When xenografts were established, four study arms initiated the treatments consisting of omacetaxine monotherapy, pomalidomide monotherapy, omacetaxine/pomalidomide combination therapy, and vehicle control IP daily Monday-Friday (FIG. 9C). Based on BLI monitoring, the disease development occurred more slowly in the omacetaxine/pomalidomide combination arm (FIG. 9D). Mouse survival was most significantly extended in the combination arm compared to the vehicle control arm (FIGS. 9E and 9F).

### Example 4—Omacetaxine and Pomalidomide Produce a Double Hit on IRF4 and c-MYC

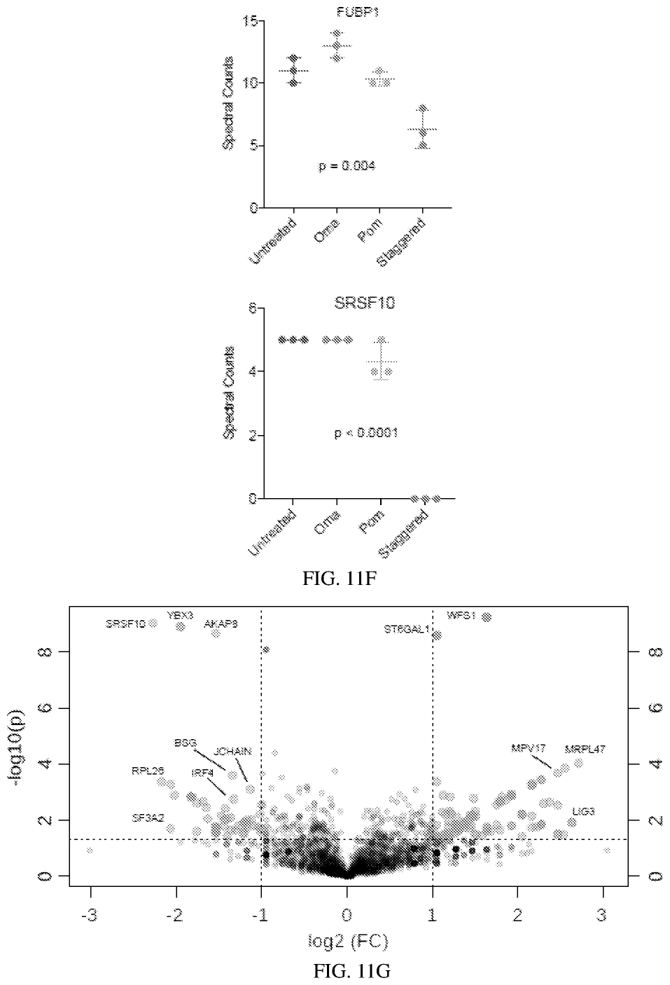

Whole cell proteomics of MM cells treated with the omacetaxine/pomalidomide combination were compared to single agent treatments at a timepoint before substantial apoptosis or cell death began. Although omacetaxine is rapidly cytotoxic to MM cells, the cytotoxicity of pomalidomide occurs after a longer delay, with apoptosis and cell death beginning at 24 h (FIG. 10A). In addition, the glycolytic capacity was reduced most drastically in MM cell lines after combination treatment (FIGS. 10B-10D). For proteomics, MM1.S cells were treated in triplicate with omacetaxine for 4 h, pomalidomide for 24 h, or the staggered combination treatment and compared to untreated controls.

The results of proteomics were analyzed for the proteins most differentially changed compared to untreated controls. By PCA analysis, the treatments separated differentially into separate groups from the control and from each other (FIG. 11A). The top 25 most differentially affected proteins are indicated by heatmap in FIG. 11B. Among the proteins most downregulated by omacetaxine single-agent treatment were IGL1 and JCHAIN, components of the IgA protein produced by the MM1.S cells (FIGS. 11C and 11D). Among the most differentially downregulated proteins by the combination treatment were IRF4, FUBP1 and SRSF10 (FIGS. 11E-11G). IRF4 stood out, as this is a known downstream target of IMiD-mediated Ikaros degradation. Furthermore, the stepwise decrease of IRF4 by treatment arm supported that the combination treatment may act as a double hit on the levels of this protein.

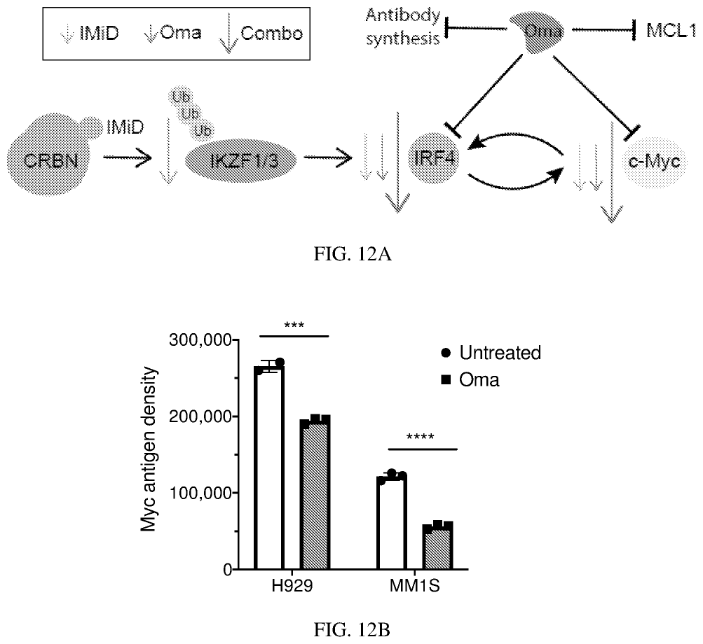

In MM, IRF4 and c-MYC forma positive feedback loop that propels malignant survival and proliferation. A model in which omacetaxine and IMiDs synergize to cause a greater reduction of IRF4 and c-MYC than either drug alone is illustrated by FIG. 12B. Confirmation of downregulation of c-MYC by omacetaxine is shown in FIG. 12A. Omacetaxine and IMiDs are synergistic in MM cells and cooperate to elicit a more substantial downregulation of the IRF4/c-MYC pathway than either drug alone, creating an attractive and clinically testable regimen for patients with relapsed/refractory MM.

### Example 5—Synergism of Both Translation Inhibitor and Immunomodulatory Drug Classes with BET Inhibitors JQ1, ABBV-075, and OTX015

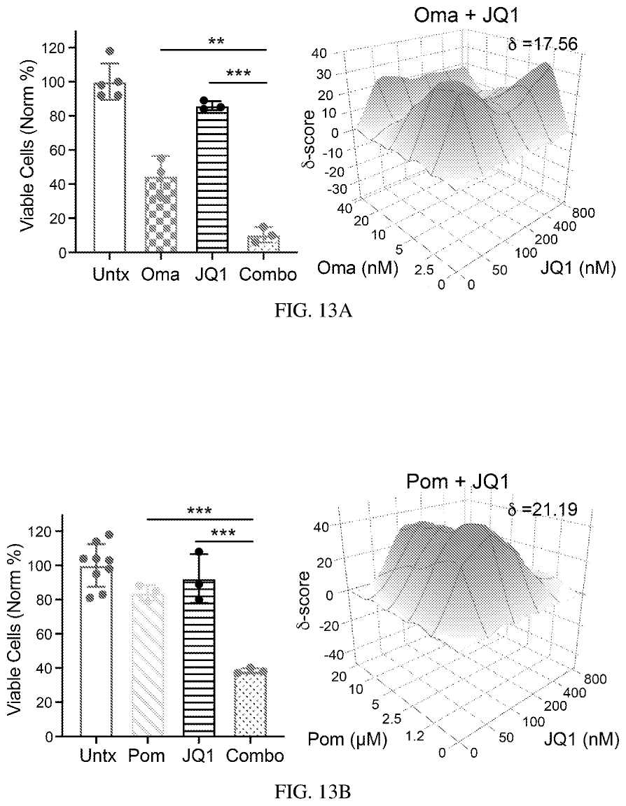

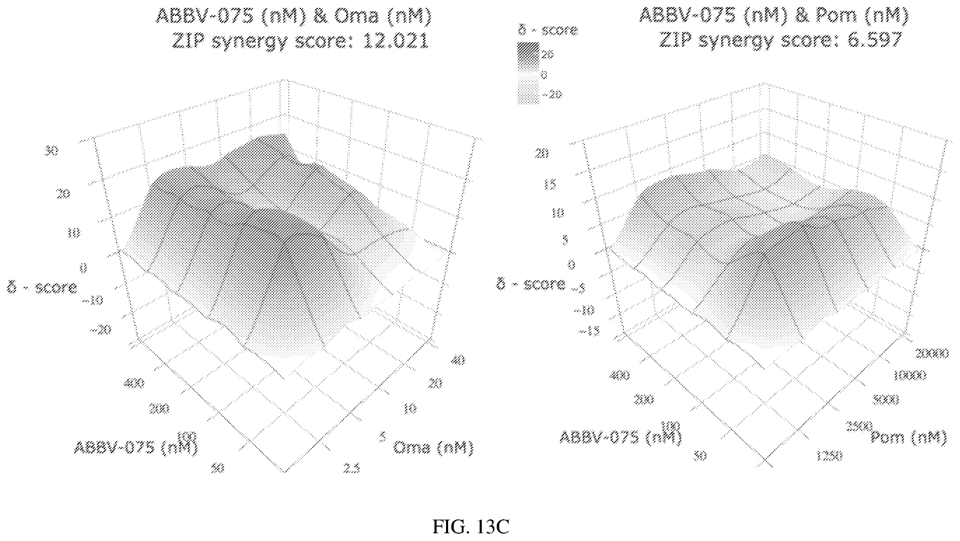

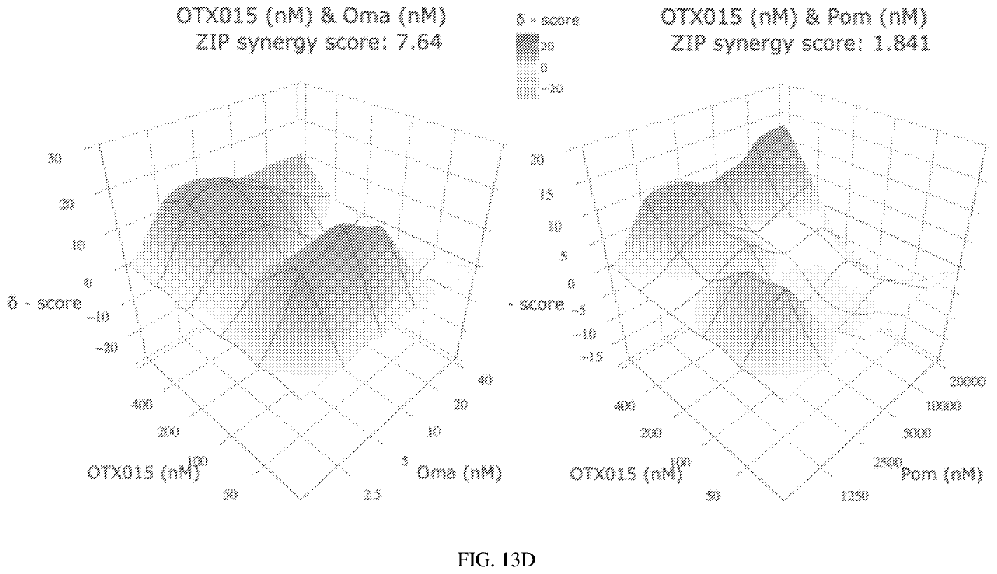

To further test the above-described synergism model, it was evaluated whether the protein translation inhibitor drug class and the immunomodulatory drug class would each be synergistic with the BET inhibitor drug class that downregulates c-MYC by the distinct mechanism of transcription regulation. As shown in FIGS. 13A and 13B, the BET inhibitor JQ1 was synergistic with both omacetaxine (FIG. 13A) and pomalidomide (FIG. 13B) in MM cell lines (17.56 and 21.18 δ-scores, respectively). Next, the BET inhibitor ABBV-075 was tested for synergism with either omacetaxine or pomalidomide. As shown in FIG. 13C, ABBV-075 was synergistic with both omacetaxine and pomalidomide in MM cell lines (12.02 and 6.597 δ-scores, respectively). In addition, the BET inhibitor OTX015 was tested for synergism with either omacetaxine or pomalidomide. As shown in FIG. 13D, OTX015 was synergistic with both omacetaxine and pomalidomide in MM cell lines (7.64 and 1.84 δ-scores, respectively).

### Example 6—Methods

### Drugs

Omacetaxine mepesuccinate/homoharringtonine (referred to herein as omacetaxine or Oma) and (+)-JQ1 were purchased from Selleckchem. The PIs, IMiDs, and dexamethasone were purchased from Thermo Fisher Scientific (Waltham, Mass.). Four-Hydroperoxy Cyclophosphamide (4-HC) was purchased from Santa Cruz Biotechnology (Dallas, Tex.), and daratumumab was purchased from the University of Colorado Health Pharmacy.

### Cell Lines

Seven different MM cell lines, including RPMI-8226, U266, NCI-H929, OPM-2, AMO-1, MM.1S, and MM.1R, were obtained from American Type Culture Collection (ATCC). Cell lines were validated via short tandem repeat polymorphism (STR) profile match analysis. Cell lines were cultured at 37° C. with 5% CO2 in RPMI 1640 medium supplemented with 10% fetal bovine serum (FBS), 100 U/mL penicillin, and 100 μg/mL streptomycin (Thermo Fisher Scientific, Asheville, N.C.).

### Cell Line Cytotoxicity

Either CellTiter-Glo Luminescent Cell Viability or CellTiter 96 AQueous MTS Cell Proliferation assay (Promega, Madison Wis.) were used to generate dose response curves of myeloma cell line proliferation treated with various anti-myeloma agents. Working solutions of CellTiter-Glo or CellTiter AQueous reagent were prepared per manufacturer instructions. Cells were combined with drug of interest at a concentration of 50,000 cells/well and incubated in 96-well culture plates at 37° C. for 96 h. After the incubation, the viability or proliferation results were obtained via Vmax Kinetic Microplate Reader (Molecular Devices, Sunnyvale Calif.). Cell line toxicity was also measured using high throughput flow cytometry. Cells were stained for viability using the LIVE/DEAD Fixable Near-IR Dead Cell Stain (Invitrogen, Carlsbad Calif.). Fifty microliters of cells from each 96-well were analyzed using a BD FACSCelesta Multicolor Cell Analyzer (BD Biosciences, San Jose Calif.) equipped with a high throughput sampler (HTS).

### Drug Combination Studies

Drug combination studies were set up using a full matrix design with myeloma cell lines or primary samples. Synergy was calculated using ZIP delta score via the SnergyFinder online tool (synergyfinder.fimm.fi). Combination index values were calculated using the CompuSyn software (ComboSyn Inc).

### Patient Sample Processing

Bone marrow aspirates were collected from patients after informed consent. Samples from patients with MM or patients with smoldering myeloma were obtained from the hematologic malignancies tissue bank. Mononuclear cells (MNCs) were isolated from the samples using SepMate Ficoll-Plaque tubes (StemCell Technologies) according to the manufacturer's instructions. Samples were cryopreserved in freezing medium consisting of Iscove's Modified Dulbecco's Medium (IMDM), 45% fetal bovine (FBS), and 10% dimethylsulfoxide (DMSO) at 10 million cells/mL.

### Ex Vivo Drug Sensitivity Testing

De-identified primary myeloma samples were thawed in a 37° C. water bath and then incubated with 100 μg DNase I (Sigma-Aldrich) for 90 sec. Samples were washed twice with 10 mL Dulbecco's Phosphate Buffered Saline without calcium and magnesium (DPBS). Cells were cultured in RPMI1640 medium containing L-glutamine with 10% FBS and 100 U/mL penicillin, 100 ug/mL streptomycin (Thermo Fisher Scientific), and 2 ng/mL interleukin 6 IL-6 (PreproTech) at 37° C. for 2 h. Cells were then transferred to 96-well plates at 4.5×10 cells/mL (90,000 MNCs per well) and treated for 48 h. Stock solutions of drug were prepared at 10 mM in dimethylsulfoxide (DMSO) and stored at −20° C. until use.

### Flow Cytometry

After 48 h treatment, the primary samples were washed in 1× cold DPBS and re-suspended in BD Brilliant Stain Buffer (BD Biosciences, San Jose Calif.) in a 96-well V-bottom plate. Cells were treated with FcR Blocking Reagent (Miltenyi Biotec, San Diego Calif.) for 5 min and then surface stained with anti-CD19-BV786 anti-CD45-BV510, anti-CD38-PerCP-Cy5.5, anti-CD138-BV421, and anti-CD319-Alexa647 or anti-CD46-Alexa647 antibodies for 10 min on ice. Intracellular staining with anti-kappa-BV605 and anti-lambda-PE light chains was performed after paraformaldehyde fixation and permeabilization. All flow cytometry antibodies were purchased from BD Biosciences. After staining, samples were washed and resuspended with 100 μl DPBS containing 2% FBS (FACS buffer). Viability staining was done using the LIVE/DEAD Fixable Near-IR Dead Cell Stain Kit (Invitrogen, Carlsbad Calif.) by adding 1 μl of 10-fold diluted stain to each well. Flow cytometry data was collected using a BD FACSCelesta Multicolor Cell Analyzer (BD Biosciences, San Jose Calif.) equipped with a HTS. For each sample a fixed volume of 175 μL was collected by the HTS. Flow cytometry data analysis was completed using FlowJo software (FlowJo LLC, Ashland Oreg.). Myeloma populations were analyzed by gating CD19− CD45+/−CD38+ CD138+ based on the cells expressing clonal light chain restriction in each sample.

Quantitative flow cytometry was used to measure MCL1 and BCL2 densities per cell. Anti-MCL1 and anti-BCL2 antibodies were labeled with Alexa Fluor 647 (Molecular Probes/life Technologies) according to manufacturer's instructions. Intracellular staining of MCL1 and BCL2 was conducted following permeabilization with the Cytofix/Cytoperm kit (BD). Mean fluorescence intensity was converted to molecules of equivalent soluble fluorochrome (MESF) by generating a standard curve with Quantum™ beads (Bangs Labs, Fishers, Ind.). The fluorophore-to-antibody ratio of the labeled antibodies was determined using Simply Cellular® anti-mouse IgG beads (Bangs Labs). Finally, conversion of MESF to cell surface antigen density was calculated by dividing by the fluorophore-to-antibody ratio.

Protein Translation Measurement

Protein synthesis levels were determined in vitro utilizing the puromycin analog O-propargyl-puromycin (OP-Puro) in the protein synthesis assay kit (Cayman Chemical) according to manufacturer instructions. For in vivo protein synthesis, engrafted NSG mice were injected IP with 500 μg puromycin (Gibco), then euthanized after 1.5 h, marrow was flushed, and cells stained with anti-puromycin antibody (12D10, Alexa-488, Millipore Sigma), followed by flow cytometry.

### BH3 Priming Assay

To evaluate BCL2 family member dependence in myeloma cell lines and patient samples, we adapted the published assay for BH3 profiling. Cells were first surfaced stained to identify markers as well as for viability. The cells were then exposed to the various BH3 mimetic peptides to test for BH3 priming, including BIM, BAD, and NOXA. Mitochondria were then permeabilized with digitonin, and the cells were fixed with paraformaldehyde. The final BH3 profile was determined using flow cytometry and staining for cytochrome-c loss.

### Myeloma Cell Line Xenografts

For in vivo assessment of omacetaxine, 5×10⁵ MM1.S cells stably expressing firefly luciferase were injected intravenously into NSG mice (Jackson Labs, Bar Harbor, Me.) to generate an orthometastatic MM xenograft model. When untreated, the NSG mice succumb to orthometastatic myeloma-like disease ˜50 days after implantation in this model. Bioluminescence imaging (BLI) was used to monitor graft status weekly. Three mouse groups were treated with omacetaxine and compared to vehicle control (PBS). Treatment was administered by intraperitoneal (IP) injection in a final volume 0.5 ml of PBS with 0.5% FBS. Tumor status was assessed by BLI, and the results were analyzed by Living Image (PerkinElmer). Following treatment, the mice were continuously monitored for survival endpoints.

### Myeloma Cell Metabolism

MM cell lines were incubated with pomalidomide for 24 h, omacetaxine for 4 h, or in staggered combination. The cells were washed and resuspended in Agilent Seahorse XF DMEM Medium, pH 7.4 (Agilent Technologies, Santa Clara, Calif.). Glycolytic flux, extracellular acidification rate (ECAR) was measured on a Seahorse XFe96 Analyzer using the Seahorse XF Glycolysis Stress Test Kit according to manufacture instructions (Agilent Technologies).

### Myeloma Cell Proteomics Analysis

Cells were lysed with RIPA buffer (Thermo Fisher Scientific) and digested according to the FASP protocol. Recovered peptides were dried, desalted, and concentrated on Thermo Scientific Pierce C18 Tips. Cell lysates were analyzed using an Orbitrap Fusion mass spectrometer connected to an EASY-nLC 1200 system (Thermo Fisher Scientific) with a nanoelectrospray ion source. Data was acquired using the Xcaliburm (version 4.1) software. MS/MS spectra were extracted, and Proteome Discoverer Software (ver. 2.1.0.62) was used to convert the raw data into mgf files. The files were then searched against a human database using an in-house Mascot server (Version 2.6, Matrix Science). Scaffold (version 4.8, Proteome Software, Portland, Oreg.) was used to validate MS/MS based peptide and protein identifications. Protein identifications were established using a greater than 99.0% probability cutoff and contained at least two identified unique peptides. Heatmaps and principal component analysis (PCA) were generated using MetaboAnalyst software (McGill University, Quebec Canada).

### Statistical Analysis

Statistics and figures were generated using Prism 6 software (GraphPad Software, San Diego, Calif.). All data are presented as mean and standard deviation. Two-tailed Student's t test was used for comparing two means. When comparing more than two means, ANOVA was used with Tukey's correction. Survival analysis was conducted using SAS version 9.4 (SAS Institute) with Cox proportional hazard models to calculate hazard ratios (HRs). Levels of statistical significance are shown by: *-p<0.05, **-p<0.01, ***-p<0.001, ****-p<0.0001.

1. A method of treating multiple myeloma in a subject, comprising administering to the subject:
i) a therapeutically effective amount of at least one protein translation inhibitor and a therapeutically effective amount of at least one immunomodulatory drug (IMiD);
ii) a therapeutically effective amount of at least one protein translation inhibitor and a therapeutically effective amount of at least one bromodomain extra-terminal (BET) inhibitor; or
iii) a therapeutically effective amount of at least one IMiD and a therapeutically effective amount of at least one BET inhibitor.

2. (canceled)

3. The method of claim 1, wherein:
i) a first pharmaceutical composition or group of pharmaceutical compositions comprises the therapeutically effective amount of the at least one protein translator inhibitor, and a second pharmaceutical composition or group of pharmaceutical compositions comprises the at least one IMiD;
ii) a first pharmaceutical composition or group of pharmaceutical compositions comprises the therapeutically effective amount of the at least one protein translator inhibitor, and a second pharmaceutical composition or group of pharmaceutical compositions comprises the at least one BET inhibitor; or
iii) a first pharmaceutical composition or group of pharmaceutical compositions comprises the therapeutically effective amount of the at least one IMiD, and a second pharmaceutical composition or group of pharmaceutical compositions comprises the at least one BET inhibitor.

4. The method of claim 1, wherein a single pharmaceutical composition comprises:
i) the therapeutically effective amount of the at least one protein translation inhibitor and the therapeutically effective amount of the at least one IMiD;
ii) the therapeutically effective amount of the at least one protein translation inhibitor and the therapeutically effective amount of the at least one BET inhibitor, or
iii) the therapeutically effective amount of the at least one IMiD and the therapeutically effective amount of the at least one BET inhibitor.

5. The method of claim 1, further comprising administering to the subject a therapeutically effective amount of at least one additional agent that is:
i) neither a protein translation inhibitor or an IMiD wherein a protein translation inhibitor and an IMiD are administered to the subject;
ii) neither a protein translation inhibitor or a BET inhibitor wherein a protein translation inhibitor and a BET inhibitor are administered to the subject; or
iii) neither an IMiD or a BET inhibitor wherein an IMiD and a BET inhibitor are administered to the subject.

6. The method of claim 5, wherein the at least one additional agent is selected from the group of: dexamethasone, prednisone, prednisolone, methylprednisolone, hydrocortisone, bortezomib, carfilzomib, marizomib, ixazomib, oprozomib, bendamustine, carmustine, cyclophosphamide, melphalan, melphalan hydrochloride, afuresertib, ibrutinib, dinaciclib, panobinostat, rocilinostat, vorinostat, siltuximab, filanesib, daratumumab, elotuzumab, indatuximab, SAR650984, doxorubicin hydrochloride, panobinostat, plerixafor, and selinexor.

7. The method of claim 1, wherein:
the at least one protein translation inhibitor is selected from the group of: omacetaxine mepesuccinate, anisomycin, lactimidomycin, cycloheximide, verrucarin A, cephaeline, emetine, bouvardin, puromycin, didemnins, and bruceantin;
the at least on IMiD is selected from the group of: lenalidomide, pomalidomide, thalidomide, and iberdomide; and
the at least one BET inhibitor is selected from the group of: JQ1, ABBV-075, FT-1101, GSK525762 (I-BET762), INCB057643, ZEN003694, OTX015 (MK-8628), GSK2820151(I-BET151), CC-90010, CPI-0610, PLX51107, ABBV-744, BI 894999, BMS-986158, GS-5829, INCB054329, and RO6870810 (TEN-010).

8. The method of claim 1, wherein the at least one protein translation inhibitor comprises omacetaxine mepesuccinate, and the therapeutically effective amount of omacetaxine mepesuccinate inhibitor is about 1.25 mg/m².

9. The method of claim 1, wherein the at least one protein translation inhibitor comprises lenalidomide, and the therapeutically effective amount of lenalidomide is about 2.5 mg to about 25 mg.

10. The method of claim 1, wherein the at least one IMiD comprises pomalidomide, and the therapeutically effective amount of pomalidomide is about 1 mg to about 4 mg.

11. The method of claim 1, wherein the at least one IMiD comprises thalidomide, and the therapeutically effective amount of thalidomide is about 200 mg.

12. The method of claim 1, wherein the at least one IMiD comprises iberdomide, and the therapeutically effective amount of iberdomide is about 0.3 mg to about 1.2 mg.

13. The method of claim 1, wherein the subject is resistant to one or more multiple myeloma therapeutic agents.

14. The method of claim 13, wherein the subject is resistant to one or more multiple myeloma therapeutic agents selected from: an IMiD, a proteasome inhibitor, and an antibody.

15. A method of treating multiple myeloma in a subject, comprising administering to the subject a therapeutically effective amount of at least one protein translation inhibitor.

16. The method of claim 15, wherein the at least one protein translation inhibitor is selected from the group of: omacetaxine mepesuccinate, anisomycin, and lactimidomycin.

17. The method of claim 16, wherein the at least one protein translation inhibitor does not comprise omacetaxine mepesuccinate, or is not omacetaxine mepesuccinate.

18. The method of claim 15, wherein the at least one protein translation inhibitor comprises omacetaxine mepesuccinate, and the therapeutically effective amount of omacetaxine mepesuccinate inhibitor is about 1.25 mg/m².

19. The method of claim 15, further comprising administering to the subject a therapeutically effective amount of at least one additional agent that is not a protein translation inhibitor.

20. The method of claim 19, wherein the at least one agent is selected from the group of: lenalidomide, pomalidomide, thalidomide, iberdomide, dexamethasone, prednisone, prednisolone, methylprednisolone, hydrocortisone, bortezomib, carfilzomib, marizomib, ixazomib, oprozomib, bendamustine, carmustine, cyclophosphamide, melphalan, melphalan hydrochloride, afuresertib, ibrutinib, dinaciclib, panobinostat, rocilinostat, vorinostat, siltuximab, filanesib, daratumumab, elotuzumab, indatuximab, SAR650984, doxorubicin hydrochloride, panobinostat, plerixafor, selinexor, JQ1, ABBV-075, FT-1101, GSK525762 (I-BET762), INCB057643, ZEN003694, OTX015 (MK-8628), GSK2820151 (I-BET151), CC-90010, CPI-0610, PLX51107, ABBV-744, BI 894999, BMS-986158, GS-5829, INCB054329, and RO6870810 (TEN-010).

21. The method of claim 15, wherein the subject is resistant to one or more anti-multiple myeloma agents.

22. (canceled)
